(19) United States
(12) Patent Application Publication
(10) Pub. No.: US 2017/0100411 A1
(43) Pub. Date: Apr. 13, 2017

(54) CORTICOSTEROID FORMULATIONS FOR MAINTAINING CORTICOSTEROID SYNOVIAL FLUID CONCENTRATIONS

(71) Applicant: Flexion Therapeutics, Inc., Burlington, MA (US)

(72) Inventors: Neil Bodick, Burlington, MA (US); Derek Jackson, Burlington, MA (US); Toni Williamson, Burlington, MA (US)

(21) Appl. No.: 15/292,670

(22) Filed: Oct. 13, 2016

Related U.S. Application Data
(60) Provisional application No. 62/240,811, filed on Oct. 13, 2015.

(51) Int. Cl. A61K 31/56; A61K 31/58; A61K 31/573; A61K 9/00

(52) U.S. Cl. CPC A61K 31/56; A61K 9/0019; A61K 31/58; A61K 31/573

(57) ABSTRACT

This invention relates to compositions and methods for achieving and maintaining maximal analgesic effect following intra-articular administration of corticosteroid formulations. The invention also describes extended release, e.g., controlled- or sustained-release corticosteroid formulations, including extended release, e.g., controlled- or sustained-release formulations of triamcinolone acetonide (TCA), fluticasone propionate, cortisol, ciclesonide (monopropionate), beclometasone diproprionate, dexamethasone, flunisolide, budesonide, desisobutyryl-ciclesonide, and/or mometasone furoate, that produce a maximal analgesic effect greater than the acute analgesic effect provided by standard corticosteroid suspensions, including non-extended release corticosteroid suspensions, and that are also associated with a clinically insignificant effect on endogenous cortisol production following administration, for example, intra-articular, intrathecal, epidural, intra-bursal, or other local administration.

### RELATED APPLICATIONS

This application claims the benefit of U.S. Provisional Application No. 62/240,811, filed Oct. 13, 2015, the contents of which are incorporated herein by reference in their entirety.

### FIELD OF THE INVENTION

This invention relates to compositions and methods for achieving and maintaining maximal analgesic effect following intra-articular administration of corticosteroid formulations. The invention also describes controlled- or sustained-release corticosteroid formulations, including controlled- or sustained-release formulations of triamcinolone acetonide (TCA), fluticasone propionate, cortisol, ciclesonide (monopropionate), beclometasone diproprionate, dexamethasone, flunisolide, budesonide, desisobutyryl-ciclesonide, and/or mometasone furoate, that produce a maximal analgesic effect greater than the acute analgesic effect provided by standard corticosteroid suspensions, including non-extended release corticosteroid suspensions, and that are also associated with a clinically insignificant effect on endogenous cortisol production following administration, for example, intra-articular, intrathecal, epidural, intra-bursal, or other local administration.

### BACKGROUND OF THE INVENTION

Corticosteroids influence all tissues of the body and produce various cellular effects. These steroids regulate carbohydrate, lipid, protein biosynthesis and metabolism, and water and electrolyte balance. Corticosteroids influencing cellular biosynthesis or metabolism are referred to as glucocorticoids while those affecting water and electrolyte balance are mineralocorticoids. Both glucocorticoids and mineralocorticoids are released from the cortex of the adrenal gland.

Corticosteroids are used in the treatment of a variety of indications, including osteoarthritis (OA) and other disorders and diseases associated with joint pain and/or inflammation. OA is a growing worldwide epidemic. In the United States alone, OA is the most common joint disease, affecting approximately 27 million Americans, with numbers expected to grow as a result of aging, obesity and sports injuries.

However, current therapies for OA are suboptimal. Oral drugs, while they may offer adequate analgesia for early-stage OA pain, are associated with serious side effects such as gastrointestinal bleeding and cardiovascular events, and, importantly, are eventually ineffective at managing OA pain as the disease progresses. Moreover, current IA therapies may be well-tolerated, but they provide pain relief that is insufficient or inadequate in duration. All IA therapies approved for OA are non-extended release suspensions or solutions that leave the joint within hours to days and are absorbed systemically, which may result in undesirable side effects.

Accordingly, there is a medical need for IA therapies for joint pain and/or inflammation that provide maximal analgesic effect over a prolonged duration and minimize undesirable side effects.

### SUMMARY OF THE INVENTION

Described herein are compositions and methods for achieving and maintaining maximal analgesic effect over a prolonged duration following intra-articular (IA) administration of a corticosteroid formulation. These compositions and methods are useful in patients with osteoarthritis (OA) and other diseases and disorders associated with joint pain and/or inflammation.

In some embodiments, the compositions are extended release formulations in which the corticosteroid is released over a prolonged period of time. In some embodiments, the compositions are extended release formulations in which the corticosteroid is released for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, and/or at least 24 weeks or more.

In some embodiments, the extended release formulations are sustained- or controlled-release formulations in which the corticosteroid is released at a uniform or substantially uniform rate over a prolonged period of time. In some embodiments, the extended release formulations are sustained- or controlled-release formulations in which the corticosteroid is released at a uniform or substantially uniform rate for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, and/or at least 24 weeks or more.

In a previously reported double-blind study, 228 patients with moderate to severe knee OA pain were randomized to an IA injection of a sustained release dosage form (containing 10, 40, or 60 mg TCA) or 40 mg of a non-extended release suspension of triamcinolone acetonide (TCA IR). Average Daily Pain (ADP) scores on the 11-point Numeric Rating Scale (NRS) were collected over 12 weeks. The 40 mg dose of the TCA sustained release dosage form produced pain relief superior to TCA IR at Weeks 5-10, and between Weeks 2 and 12, the magnitude of the analgesic effect of the 40 mg dose of the TCA sustained release dosage form exceeded the maximum observed effect of 40 mg TCA IR at Week 4 (FIG. 1C). Of note, the acute effect of TCA IR is among the largest reported analgesic effects in OA of the knee (see e.g., Bjordal J M, Johnson M I, Lopes-Martins R A B, Bogen B, Chow R, Ljunggren A E. Short-term efficacy of physical interventions in osteoarthritic knee pain. A systematic review and meta-analysis of randomised placebo-controlled trials. BMC Musculoskelet Disord 2007; 8:51), and the amplification of this analgesic signal using an extended release dosage form, for example, a sustained release dosage form, is a novel observation. The compositions and methods provided herein provide extended release of a corticosteroid and maintain the synovial concentration of the corticosteroid at a level that amplifies the analgesic signal.

The compositions and methods exploit this novel observation by maximizing analgesic effect and maintaining this maximal analgesic effect over a prolonged duration in a patient with a disease or disorder associated with joint pain and/or joint inflammation. The compositions and methods maximizing analgesic effect and maintaining this maximal analgesic effect over a prolonged duration by administering to a subject in need thereof a corticosteroid at a dosage sufficient to produce a desired synovial concentration of the corticosteroid for a duration of at least one week, e.g., at least two weeks, at least three weeks, at least one month, at least two months, at least three months, at least four months, at least five months, at least six months, at least seven months, at least eight months, at least nine months, at least ten months, at least 11 months, or at least 12 months or beyond. The ability to maintain this desired synovial concentration of the corticosteroid can be accomplished through the use of an extended release formulation such as, for example, a controlled- or sustained-release formulation comprising the corticosteroid, by one or more injections of the corticosteroid, either in a non-extended release formulation or suspension, or in an extended release formulation such as, e.g., a controlled- or sustained-release formulation, or a combination thereof.

In some embodiments, the compositions are extended release formulations in which the corticosteroid is released over a prolonged period of time. In some embodiments, the compositions are extended release formulations in which the corticosteroid is released for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, and/or at least 24 weeks or more.

In some embodiments, the extended release formulations are sustained- or controlled-release formulations in which the corticosteroid is released at a uniform or substantially uniform rate over a prolonged period of time. In some embodiments, the extended release formulations are sustained- or controlled-release formulations in which the corticosteroid is released at a uniform or substantially uniform rate for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, and/or at least 24 weeks or more.

The compositions and methods of the disclosure are based on the first studies to detect and determine synovial fluid concentration of an intra-articular (IA) corticosteroid in patients following IA injection. Furthermore, these studies are the first to demonstrate that administration of an extended release formulation such as, e.g., a sustained-release corticosteroid formulation maintains a synovial fluid concentration of the corticosteroid at a level that produces an analgesic effect that is greater than the maximum analgesic effect observed for the non-extended release formulation, e.g., a non-extended release suspension formulation, for the corresponding corticosteroid. The compositions and methods of the disclosure achieve and maintain this improved level of analgesic effect in the patient for a sustained period of time, e.g., at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days or longer, at least four months or longer, at least five months or longer, at least six months or longer, at least seven months or longer, at least eight months or longer, at least nine months or longer, at least 10 months or longer, at least 11 months or longer, or at least 12 months or longer. In these studies, the synovial fluid concentration of TCA following administration of non-extended release TCA (referred to herein as “TCA-IR”), the concentration of free drug in synovial fluid at intervals such as 6 weeks post-administration or 12 weeks post-administration was below the level of quantification. A non-extended release corticosteroid formulation is a formulation or suspension in which most or all of the corticosteroid is released within at least four weeks post-administration, within at least three weeks post-administration, within at least two weeks post-administration, or within at least one week post-administration.

These studies identify a range of synovial fluid concentration of a corticosteroid that produces maximal analgesic effect when maintained for a period of approximately 4 to 6 weeks post administration. Further, if this range of synovial fluid concentration is maintained for periods exceeding 4 to 6 weeks, this maximal analgesic effect will persist. In some embodiments, the synovial concentration is maintained for at least 3 months. In some embodiments, the synovial concentration is maintained for at least 6 months. In some embodiments, the synovial concentration is maintained for between at least 3 months and 12 months. In some embodiments, the synovial concentration is maintained for between at least 3 months and 6 months. In some embodiments, the synovial concentration is maintained for between at least 6 months and 12 months.

This discovery is a broadly applicable breakthrough with wide-ranging implications. Importantly, the identification of a synovial fluid concentration range for administered corticosteroids is independent of the formulation and/or dosing used, provided that the formulation is effective to deliver the corticosteroid over an extended period of time and to maintain a synovial fluid concentration of corticosteroid in the desired range.

Analgesic effect is assessed using any of a variety of art-recognized methods for evaluating analgesic effect. For example, analgesic effect can be assessed using weekly average of daily (24 hour) pain intensity score, Western Ontario & McMaster University Osteoarthritis Index (WOMAC®, available on the WOMAC website) and patient and clinical global impression of change. Analgesic effect can also be assessed by monitoring evidence of inflammation in a patient. For example, evidence of inflammation can be assessed by monitoring signs of local inflammation including tenderness, swelling, redness/heat, effusion, and Baker's cyst, and synovitis (as assessed with imaging technologies) at various time intervals.

These studies identify a range of synovial fluid concentration of a corticosteroid that produces and maintains maximal analgesic effect in a patient for an extended period of time post administration. This discovery is a broadly applicable breakthrough with wide-ranging implications. Importantly, the identification of a synovial fluid concentration range for administered corticosteroids is independent of the formulation and/or dosing used, provided that the formulation is effective to deliver the corticosteroid over an extended period of time and to maintain a synovial fluid concentration of corticosteroid in the desired range.

The first set of examples provided herein use TCA, but this discovery is not limited to TCA or even to Class B corticosteroids in general. The second set of examples demonstrates the ability of an extended release formulation such as, e.g., a controlled- or sustained-release formulation, comprising fluticasone propionate and a PLGA polymer to achieve and maintain the desired synovial concentration of fluticasone propionate. For other corticosteroids, the relative potency and protein binding of various corticosteroids are known in the art (See e.g., Derendorf H, Nave R, Drollmann A, Cerasoli F, Wurst W. Relevance of pharmacokinetics and pharmacodynamics of inhaled corticosteroids to asthma. Eur Respir J 2006; 28:1042-1050; Chan W L, Carrell R W, Zhou A, Read R J. How Changes in Affinity of Corticosteroid-binding Globulin Modulate Free Cortisol Concentration. J Clin Endocrinol Metab 2013 August; 98(8):3315-3322; Wilkinson M and Jones B S. Electrophoretic Studies of Synovial Fluid Proteins. Ann Rheum Dis 1964; 23:22). As such, one of ordinary skill in the art would be able to use the data and information gathered for the post-administration synovial fluid concentration of TCA to calculate and determine the appropriate range of synovial fluid concentration needed to achieve and maintain maximal analgesic effect over a prolonged duration in a patient for any given corticosteroid.

The first set of working examples provided herein demonstrate the detection and quantification of synovial fluid concentration of TCA following administration of a TCA/PLGA microparticle formulation referred to herein as “TCA Formulation 1” (TCA Form. 1) and standard, non-extended release TCA suspension (TCA IR) at doses known to have analgesic effect. “TCA Formulation 1” is an extended-release formulation of triamcinolone acetonide (TCA) in poly (lactic-co-glycolic acid) (PLGA) microspheres that is designed to maintain therapeutic concentrations of TCA in the joint over a period of approximately 3 months.

FIG. 1C demonstrates that 40 mg dose of a TCA/PLGA formulation achieves maximal analgesic effect around 6-8 weeks post IA administration. However, this analgesic effect begins to decrease thereafter as the synovial fluid concentration of the TCA is no longer present within the ranges disclosed herein, as evidenced by the level of analgesic effect at 12 weeks in FIG. 1C.

The second set of working examples provided herein demonstrate the detection and quantification of synovial fluid concentration of fluticasone propionate following administration of a fluticasone propionate PLGA microparticle formulation referred to herein as a “FP/PLGA formulation.” FIGS. 2A and 2B demonstrate that a single 5 mg/mL intra-articular injection of the FP/PLGA formulation maintains sustained plasma and synovial fluid concentration of fluticasone propionate for at least six months post-dosing.

Thus, the corticosteroid formulations suitable for use in the compositions and methods of the disclosure include any corticosteroid formulation that produces and maintains the synovial fluid concentration of corticosteroid that produces maximal analgesic effect for a sustained period of time, e.g., at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer. As used herein, the term “maximal analgesic effect” and variations thereof is a level of analgesic effect, observed after administration of a formulation of the disclosure, which is greater than the acute analgesic effect provided by standard, non-extended release corticosteroid suspensions.

For TCA in particular, this synovial fluid concentration is maintained at a level that is at least 6 ng/ml for a duration of at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer.

In some embodiments, the synovial fluid concentration of TCA is maintained at a level that is at least 10 ng/ml for a duration of at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer.

In some embodiments, the synovial fluid concentration of TCA is maintained at a level that is at least 15 ng/ml for a duration of at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer.

In some embodiments, the synovial fluid concentration of TCA is maintained at a level that is at least 20 ng/ml for a duration of at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer.

While suitable formulations of the disclosure include any formulation that achieves and maintains a synovial fluid concentration in the desired range, e.g., a range of at least 6 ng/ml for TCA, formulations that achieve and maintain a synovial fluid concentration significantly higher than the desired level, e.g. above an upper level of around 1000 ng/ml for TCA, will become less effective as a result of unwanted side effects of administering and/or maintaining such high levels of corticosteroid concentrations. For example, it is known that higher systemic concentrations of corticosteroid can lead to suppression of the hypothalamic-pituitary-adrenal (HPA) axis, leading to a variety of unwanted side effects. Thus, in some embodiments, the formulations provided herein achieve and maintain sufficiently high synovial fluid concentrations of corticosteroid to produce a maximal analgesic effect over a prolonged duration, while simultaneously producing sufficiently low systemic concentrations of corticosteroid to avoid adverse suppression of the HPA axis. As used herein, “adverse” suppression of the HPA axis refers to levels of cortisol suppression greater than 40%, preferably greater than 35% by day 14 post-administration, for example post-injection.

Ranges of synovial fluid concentrations are provided throughout the disclosure. Those of ordinary skill in the art will appreciate that these ranges are values based on samples from various subjects, e.g., the mean value from the detected levels at which the patients reported maximal analgesic effect. These values may vary slightly from sample to sample. Thus, the ranges of synovial concentrations provided herein are a target synovial fluid concentration, and patients having synovial fluid concentrations slightly outside the ranges provided herein, e.g., slightly below 6 ng/ml and/or slightly above 1,000 ng/ml for TCA, may still achieve and maintain maximal analgesic effect over a prolonged duration.

In some embodiments, the TCA synovial fluid concentration is in the range from about 6 ng/ml to about 1000 ng/ml. In some embodiments, the TCA synovial fluid concentration is in the range from about 6 ng/ml to about 900 ng/ml, from about 6 ng/ml to about 800 ng/ml, about 6 ng/ml to about 700 ng/ml, about 6 ng/ml to about 600 ng/ml, about 6 ng/ml to about 500 ng/ml, about 6 ng/ml to about 400 ng/ml, about 6 ng/ml to about 300 ng/ml, about 6 ng/ml to about 200 ng/ml, and/or about 6 ng/ml to about 100 ng/ml.

In some embodiments, the TCA synovial fluid concentration is in the range from about 10 ng/ml to about 1000 ng/ml. In some embodiments, the TCA synovial fluid concentration is in the range from about 10 ng/ml to about 900 ng/ml, from about 10 ng/ml to about 800 ng/ml, about 10 ng/ml to about 700 ng/ml, about 10 ng/ml to about 600 ng/ml, about 10 ng/ml to about 500 ng/ml, about 10 ng/ml to about 400 ng/ml, about 10 ng/ml to about 300 ng/ml, about 10 ng/ml to about 200 ng/ml, and/or about 10 ng/ml to about 100 ng/ml.

In some embodiments, the TCA synovial fluid concentration is in the range from about 15 ng/ml to about 1000 ng/ml. In some embodiments, the TCA synovial fluid concentration is in the range from about 15 ng/ml to about 900 ng/ml, from about 15 ng/ml to about 800 ng/ml, about 15 ng/ml to about 700 ng/ml, about 15 ng/ml to about 600 ng/ml, about 15 ng/ml to about 500 ng/ml, about 15 ng/ml to about 400 ng/ml, about 15 ng/ml to about 300 ng/ml, about 15 ng/ml to about 200 ng/ml, and/or about 15 ng/ml to about 100 ng/ml.

In some embodiments, the TCA synovial fluid concentration is in the range from about 20 ng/ml to about 1000 ng/ml. In some embodiments, the TCA synovial fluid concentration is in the range from about 20 ng/ml to about 900 ng/ml, from about 20 ng/ml to about 800 ng/ml, about 20 ng/ml to about 700 ng/ml, about 20 ng/ml to about 600 ng/ml, about 20 ng/ml to about 500 ng/ml, about 20 ng/ml to about 400 ng/ml, about 20 ng/ml to about 300 ng/ml, about 20 ng/ml to about 200 ng/ml, and/or about 20 ng/ml to about 100 ng/ml.

In some embodiments, the TCA synovial fluid concentration is in the range from about 6 ng/ml to about 78 ng/ml. In some embodiments, the TCA synovial fluid concentration is in the range from about 6 ng/ml to about 70 ng/ml, from about 6 ng/ml to about 65 ng/ml, from about 6 ng/ml to about 60 ng/ml, from about 6 ng/ml to about 55 ng/ml, from about 6 ng/ml to about 50 ng/ml, from about 6 ng/ml to about 45 ng/ml, from about 6 ng/ml to about 40 ng/ml, from about 6 ng/ml to about 35 ng/ml, from about 6 ng/ml to about 30 ng/ml, from about 6 ng/ml to about 25 ng/ml, from about 6 ng/ml to about 20 ng/ml, from about 6 ng/ml to about 15 ng/ml, and/or from about 6 ng/ml to about 10 ng/ml.

In some embodiments, the TCA synovial fluid concentration is in the range from about 10 ng/ml to about 78 ng/ml. In some embodiments, the TCA synovial fluid concentration is in the range from about 10 ng/ml to about 70 ng/ml, from about 10 ng/ml to about 65 ng/ml, from about 10 ng/ml to about 60 ng/ml, from about 10 ng/ml to about 55 ng/ml, from about 10 ng/ml to about 50 ng/ml, from about 10 ng/ml to about 45 ng/ml, from about 10 ng/ml to about 40 ng/ml, from about 10 ng/ml to about 35 ng/ml, from about 10 ng/ml to about 30 ng/ml, from about 10 ng/ml to about 25 ng/ml, from about 10 ng/ml to about 20 ng/ml, and/or from about 10 ng/ml to about 15 ng/ml.

In some embodiments, the TCA synovial fluid concentration is in the range from about 15 ng/ml to about 78 ng/ml. In some embodiments, the TCA synovial fluid concentration is in the range from about 15 ng/ml to about 70 ng/ml, from about 15 ng/ml to about 65 ng/ml, from about 15 ng/ml to about 60 ng/ml, from about 15 ng/ml to about 55 ng/ml, from about 15 ng/ml to about 50 ng/ml, from about 15 ng/ml to about 45 ng/ml, from about 15 ng/ml to about 40 ng/ml, from about 15 ng/ml to about 35 ng/ml, from about 15 ng/ml to about 30 ng/ml, from about 15 ng/ml to about 25 ng/ml, and/or from about 15 ng/ml to about 20 ng/ml.

In some embodiments, the TCA synovial fluid concentration is in the range from about 20 ng/ml to about 78 ng/ml. In some embodiments, the TCA synovial fluid concentration is in the range from about 20 ng/ml to about 70 ng/ml, from about 20 ng/ml to about 65 ng/ml, from about 20 ng/ml to about 60 ng/ml, from about 20 ng/ml to about 55 ng/ml, from about 20 ng/ml to about 50 ng/ml, from about 20 ng/ml to about 45 ng/ml, from about 20 ng/ml to about 40 ng/ml, from about 20 ng/ml to about 35 ng/ml, from about 20 ng/ml to about 30 ng/ml, and/or from about 20 ng/ml to about 25 ng/ml.

It is of note that the lower boundary of at least about 6 ng/ml is not expected, as this level is larger than the concentration known to be associated with maximal pharmacologic effect in other systems. For example, these experiments also demonstrate that the maximal effect on the HPA axis is associated with plasma concentrations of approximately 1.4 ng/ml.

The range for other corticosteroids, including, for example, a class A corticosteroid, a class B corticosteroid, a class C corticosteroid, and/or a class D corticosteroid, is calculated using the known potency and/or protein binding for any given corticosteroid, and using the TCA synovial fluid concentration range to extrapolate the range for the other corticosteroid.

In some embodiments, the corticosteroid is cortisol, and the synovial fluid concentration of cortisol is in the range of about 144.98 ng/ml to about 24.16 μg/ml, or any value in between the range of about 144.98 ng/ml to about 24.16 μg/ml.

In some embodiments, the corticosteroid is ciclesonide (monopropionate), and the synovial fluid concentration of ciclesonide (monopropionate) is in the range of about 168.93 ng/ml to about 28.15 μg/ml, or any value in between the range of about 168.93 ng/ml to about 28.15 μg/ml.

In some embodiments, the corticosteroid is beclometasone diproprionate, and the synovial fluid concentration of beclometasone diproprionate is in the range of about 32.07 ng/ml to about 5344.96 ng/ml, or any value in between the range of about 32.07 ng/ml to about 5344.96 ng/ml.

In some embodiments, the corticosteroid is dexamethasone, and the synovial fluid concentration of dexamethasone is in the range of about 14.63 ng/ml to about 2438.88 ng/ml, or any value in between the range of about 14.63 ng/ml to about 2438.88 ng/ml.

In some embodiments, the corticosteroid is flunisolide, and the synovial fluid concentration of flunisolide is in the range of about 8.63 ng/ml to about 1438.27 ng/ml, or any value in between the range of about 8.63 ng/ml to about 1438.27 ng/ml.

In some embodiments, the corticosteroid is budesonide, and the synovial fluid concentration of budesonide is in the range of about 1.84 ng/ml to about 307.11 ng/ml, or any value in between the range of about 1.84 ng/ml to about 307.11 ng/ml.

In some embodiments, the corticosteroid is desisobutyryl-ciclesonide, and the synovial fluid concentration of desisobutyryl-ciclesonide is in the range of about 1.69 ng/ml to about 281.55 ng/ml, or any value in between the range of about 1.69 ng/ml to about 281.55 ng/ml.

In some embodiments, the corticosteroid is fluticasone propionate, and the synovial fluid concentration of fluticasone propionate is in the range of about 0.95 ng/ml to about 157.58 ng/ml, or any value in between the range of about 0.95 ng/ml to about 157.58 ng/ml.

In some embodiments, the corticosteroid is mometasone furoate, and the synovial fluid concentration of mometasone furoate is in the range of about 0.77 ng/ml to about 128.93 ng/ml, or any value in between the range of about 0.77 ng/ml to about 128.93 ng/ml.

In some embodiments, the desired synovial fluid concentration of the corticosteroid is maintained by administering at least one additional dose of the corticosteroid. In some embodiments, at least one additional dose of corticosteroid is administered as an extended release formulation. In some embodiments, at least one additional dose of corticosteroid is administered as a controlled- or sustained-release formulation.

In some embodiments, the corticosteroid is released from the formulation for a duration of at least between 14 days and 90 days. In some embodiments, the corticosteroid is released from the formulation for a duration of at least between 30 days and 90 days. In some embodiments, the corticosteroid is released from the formulation for a duration of at least 3 months. In some embodiments, the corticosteroid is released from the formulation for a duration of at least between 3 months and 12 months. In some embodiments, the corticosteroid is released from the formulation for a duration of at least between 3 months and 6 months. In some embodiments, the corticosteroid is released from the formulation for a duration of at least between 6 months and 12 months.

In some embodiments, the formulation releases corticosteroid for at least 14 days at a rate that does not adversely suppress the hypothalamic-pituitary-adrenal axis (HPA axis).

In some embodiments, the formulation is administered as one or more injections. In some embodiments, the injection is one or more local injections at a site of pain. In some embodiments, the injection is one or more intra-articular or peri-articular injections.

In some embodiments, the patient has osteoarthritis, rheumatoid arthritis, acute gouty arthritis, and/or synovitis.

The corticosteroid formulations provided herein are effective at treating pain and/or inflammation with minimal long-term side effects of corticosteroid administration. In some embodiments, the corticosteroid formulations provided herein maintain sufficiently high synovial fluid concentrations of corticosteroid to be effective at treating pain and/or inflammation while producing sufficiently low systemic concentrations of corticosteroid to avoid adverse suppression of the HPA axis. In some embodiments, the corticosteroid formulations provided herein deliver the corticosteroid in a dose and in an extended release manner such as, e.g., a controlled or sustained release manner, such that the levels of cortisol suppression are at or below 40%, preferably 35% by day 14 post-administration, for example post-injection. In some embodiments, the corticosteroid formulations provided herein deliver the corticosteroid in a dose and in an extended release manner such as, e.g., a controlled or sustained release manner, such that the levels of cortisol suppression are negligible, clinically insignificant/inconsequential and/or undetectable by 14 post-administration, for example post-injection. In some embodiments, the corticosteroid formulations provided herein deliver the corticosteroid in a dose and in an extended release formulation such as, e.g., a controlled or sustained release manner, such that the levels of cortisol suppression are negligible at any time post-injection. Thus, the corticosteroid formulations in these embodiments are effective in the absence of any significant HPA axis suppression. In some embodiments, administration of the corticosteroid formulations provided herein can result in initial HPA axis suppression, for example, within the first few days, within the first two days and/or within the first 24 hours post-injection, but by day 14 post-injection, suppression of the HPA axis is less than 40%, preferably 35%.

The corticosteroid formulations are suitable for administration, for example, local administration by injection into a site at or near the site of a patient's pain and/or inflammation. In certain embodiments, a sustained release form of corticosteroid is administered locally to treat pain and inflammation. Local administration of a corticosteroid formulation can occur, for example, by injection into the intra-articular space or peri-articular space at or near the site of a patient's pain. Local administration of corticosteroid formulation can occur, for example, by intra-articular, intrathecal, epidural, or intra-bursal administration. In certain preferred embodiments of the invention, an extended release form, e.g., a sustained release form, of corticosteroid is administered (e.g., by single injection or as sequential injections) into an intra-articular space for the treatment of pain, for example, due to osteoarthritis, rheumatoid arthritis, gouty arthritis and/or other joint disorders, or into local tissues affected by bursitis, tenosynovitis, epicondylitis, synovitis, sciatica, lumbar pain, and/or other disorders. In certain embodiments of the invention, an extended release form such as, e.g., a sustained release form, of corticosteroid is administered (e.g., by single injection or as sequential injections) into an intra-articular space to slow, arrest, reverse or otherwise inhibit structural damage to tissues associated with progressive disease such as, for example, the damage to cartilage associated with progression of osteoarthritis. Because both pain and structural progression are the product of local inflammation in osteoarthritis and other disorders such are rheumatoid arthritis, and because corticosteroids act through the reduction of inflammation, the concentration ranges cited here for maximizing analgesic effect will also be effective in slowing or stopping structural progression. The corticosteroid formulations described herein are also useful in the treatment of a systemic disorder for which corticosteroid treatment would be required or otherwise therapeutically beneficial.

The corticosteroid formulations provided herein can be used in combination with any of a variety of therapeutics, also referred to herein as “co-therapies.”

For example, the corticosteroid formulations can be used in combination with a non-extended release TCA (or other corticosteroid) solution or suspension, which provides high local exposures for between 1 day and 14 days following administration and which produce systemic exposures that may be associated with transient suppression of the HPA axis. In some embodiments, the same corticosteroid, i.e., TCA, is used in both the non-extended release component and sustained release components. In some embodiments, the non-extended release component is KENALOG™ or its bioequivalent. In some embodiments, the non-extended release component contains a corticosteroid that is different from that of the extended release component, e.g., the sustained release component, i.e., the non-extended release component does not include TCA. In some embodiments, the sustained, steady state release of TCA will not adversely suppress the HPA axis.

In some embodiments, the period of extended release is between 30 days and 12 months. In some embodiments, the period of extended release is between 90 days and 12 months. In some embodiments, the period of extended release is at least at least 3 months. In some embodiments, the period of extended release is at least between 3 months and 12 months. In some embodiments, the period of extended release is at least between 3 months and 6 months. In some embodiments, the period of extended release is at least between 6 months and 12 months. In some embodiments, the period of sustained release is between 30 days and 12 months. In some embodiments, the period of sustained release is between 90 days and 12 months. In some embodiments, the period of sustained release is at least 3 months. In some embodiments, the period of sustained release is at least between 3 months and 12 months. In some embodiments, the period of sustained release is at least between 3 months and 6 months. In some embodiments, the period of sustained release is at least between 6 months and 12 months.

In some embodiments, the high local exposure attributable to the non-extended release component lasts for between 1 day and 28 days. In some embodiments, the high local exposure attributable to the non-extended release component lasts for between 1 day and 21 days. In some embodiments, the high local exposure attributable to the non-extended release component lasts for between 1 day and 14 days. In some embodiments, the high local exposure attributable to the non-extended release component lasts for between 1 day and 10 days. In some embodiments, the high local exposure attributable to the non-extended release component lasts between 1 days and 8 days. In some embodiments, the high local exposure attributable to the non-extended release component lasts between 1 days and 6 days. In some embodiments, the high local exposure attributable to the non-extended release component lasts for between 1 day and 4 days.

Suitable additional agents for use in combination with the corticosteroid formulations provided herein include hyaluronic acid preparations including but not limited to Synvisc One, Gel 200 and Supartz; NSAIDS including but not limited to aspirin, celecoxib (Celebrex), diclofenac (Voltaren), diflunisal (Dolobid), etodolac (Lodine), ibuprofen (Motrin), indomethacin (Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin); biologics including but not limited to Actemra (tocilizumab), Enbrel (etanercept), Humira (adalimumab), Kineret (anakinra), Orencia (abatacept), Remicade (infliximab), Rituxan (rituximab), Cimzia (certolizumab), and Simponi (golimumab); disease modifying agents including but not limited to methotrexate, Plaquenil (hydroxychloroquine) and Azulfidine (sulfasalazine), Minocin (minocycline); and other analgesic and anti-inflammatory agents including but not limited to p38 inhibitors JAC inhibitors, opioids, other corticosteroids, lidocaine, bupivacaine, ropivacaine, botulinum toxin A.

In some embodiments, the corticosteroid formulation and additional agent are formulated into a single therapeutic composition, and the corticosteroid formulation and additional agent are administered simultaneously. Alternatively, the corticosteroid formulation and additional agent are separate from each other, e.g., each is formulated into a separate therapeutic composition, and the corticosteroid formulation and the additional agent are administered simultaneously, or the corticosteroid formulation and the additional agent are administered at different times during a treatment regimen. For example, the corticosteroid formulation is administered prior to the administration of the additional agent, the corticosteroid formulation is administered subsequent to the administration of the additional agent, or the corticosteroid formulation and the additional agent are administered in an alternating fashion. As described herein, the corticosteroid formulation and additional agent are administered in single doses or in multiple doses.

In some embodiments, the corticosteroid formulation and the additional agent are administered by the same route. In some embodiments, the corticosteroid formulation and the additional agent are administered via different routes.

These corticosteroid formulations, preparations, and populations thereof, when administered to a patient, exhibit an improved benefit or other therapeutic outcome in the treatment of a disease, for example a joint related disorder, as compared to the administration, for example administration into the intra-articular space of a joint, of an equivalent amount of the non-extended release corticosteroid formulation or suspension, e.g., KENALOG™, absent any particulate suspension, microparticle, or other type of extended-release formulation, incorporation, admixture, or encapsulation. The improved benefit can be any of a variety of laboratory or clinical results. For example, administration of an extended-release corticosteroid formulation is considered more successful than administration of corticosteroid absent any microparticle or other extended-release formulation if, following administration of the extended-release corticosteroid formulations, one or more of the symptoms associated with the disease is alleviated, reduced, inhibited or does not progress to a further, i.e., worse, state, to a greater extent than the level that is observed after administration of corticosteroid absent any microparticle or other extended-release formulation. Administration of an extended-release corticosteroid formulation is considered more successful than administration of corticosteroid absent any microparticle or other extended-release formulation if, following administration of the extended-release corticosteroid formulations, anti-inflammatory activity is sustained for a longer period than the level that is observed after administration of corticosteroid absent any microparticle and/or any other extended-release formulation.

It is contemplated that whenever appropriate, any embodiment of the present invention can be combined with one or more other embodiments of the present invention, even though the embodiments are described under different aspects of the present invention.

### BRIEF DESCRIPTION OF THE FIGURES

FIGS. 1A, 1B, and 1C are a series of graphs depicting the results following a single administration of 40 mg TCA IR, 10 mg TCA Formulation 1 and 40 mg TCA Formulation 1. FIG. 1A depicts geometric mean of plasma concentration of TCA over 12 weeks. FIG. 1B depicts geometric mean of synovial fluid concentration at Week 6 and Week12 (bar denotes 95% confidence interval). FIG. 1C depicts weekly mean of Average Daily Pain (ADP) on the 11-point Numeric Rating Scale (NRS) (bar denotes standard error) over 12 weeks.

FIGS. 2A and 2B are a series of graphs depicting the detection of sustained plasma levels of fluticasone propionate. As shown in FIGS. 2A and 2B, fluticasone propionate was still detectable at 6 months post-dose. Each graph is shown as geometric mean (±95% CI) plasma fluticasone concentration profiles over time: linear-linear and log-linear.

FIG. 3 is a graph depicting individual, geometric mean (±95% CI), and predicted synovial fluid fluticasone concentration profiles over time: log-linear.

FIG. 4 is a graph depicting a comparison of the sustained synovial fluid drug levels for a FP/PLGA microsphere formulation as compared to the TCA/PLGA formulation referred to herein as TCA Formulation 1.

### DETAILED DESCRIPTION OF THE INVENTION

The disclosure provides compositions and methods for achieving and maintaining maximal analgesic effect over a prolonged duration following intra-articular administration of a corticosteroid formulation. These compositions and methods are useful in patients with osteoarthritis (OA) and other diseases and disorders.

The disclosure provides methods for maximizing analgesic effect and maintaining maximal analgesic effect over a prolonged duration in a patient with a disease or disorder associated with joint pain and/or joint inflammation the method by administering to a subject in need thereof an extended release formulation, such as, e.g., a controlled- or sustained-release formulation, comprising a corticosteroid, and maintaining a synovial fluid concentration of the corticosteroid that provides a pharmacological effect equivalent to a synovial fluid concentration of triamcinolone acetonide (TCA) of at least 6 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of the corticosteroid that provides a pharmacological effect equivalent to a synovial fluid concentration of TCA of at least 10 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of the corticosteroid that provides a pharmacological effect equivalent to a synovial fluid concentration of TCA of at least 15 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of the corticosteroid that provides a pharmacological effect equivalent to a synovial fluid concentration of TCA of at least 20 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

In some embodiments, the synovial fluid concentration of the corticosteroid is maintained at a synovial fluid concentration that provides a pharmacological effect equivalent to a TCA synovial fluid concentration in the range of about 6 ng/ml to about 1000 ng/ml. In some embodiments, the synovial fluid concentration of the corticosteroid is maintained at a synovial fluid concentration that provides a pharmacological effect equivalent to a TCA synovial fluid concentration in the range of about 10 ng/ml to about 1000 ng/ml. In some embodiments, the synovial fluid concentration of the corticosteroid is maintained at a synovial fluid concentration that provides a pharmacological effect equivalent to a TCA synovial fluid concentration in the range of about 15 ng/ml to about 1000 ng/ml. In some embodiments, the synovial fluid concentration of the corticosteroid is maintained at a synovial fluid concentration that provides a pharmacological effect equivalent to a TCA synovial fluid concentration in the range of about 20 ng/ml to about 1000 ng/ml.

In some embodiments, the synovial fluid concentration of the corticosteroid is maintained at a synovial fluid concentration that provides a pharmacological effect equivalent to a TCA synovial fluid concentration in the range of about 6 ng/ml to about 78 ng/ml. In some embodiments, the synovial fluid concentration of the corticosteroid is maintained at a synovial fluid concentration that provides a pharmacological effect equivalent to a TCA synovial fluid concentration in the range of about 10 ng/ml to about 78 ng/ml. In some embodiments, the synovial fluid concentration of the corticosteroid is maintained at a synovial fluid concentration that provides a pharmacological effect equivalent to a TCA synovial fluid concentration in the range of about 15 ng/ml to about 78 ng/ml. In some embodiments, the synovial fluid concentration of the corticosteroid is maintained at a synovial fluid concentration that provides a pharmacological effect equivalent to a TCA synovial fluid concentration in the range of about 20 ng/ml to about 78 ng/ml.

In some embodiments, the synovial fluid concentration of the corticosteroid is maintained for a duration of at least 50 days. In some embodiments, the synovial fluid concentration of the corticosteroid is maintained for a duration of at least 75 days. In some embodiments, the synovial fluid concentration of the corticosteroid is maintained for a duration of at least 90 days. In some embodiments, the synovial fluid concentration of the corticosteroid is maintained for a duration of at least four months or longer. In some embodiments, the synovial fluid concentration of the corticosteroid is maintained for a duration of at least five months or longer. In some embodiments, the synovial fluid concentration of the corticosteroid is maintained for a duration of at least six months or longer. In some embodiments, the synovial fluid concentration of the corticosteroid is maintained for a duration of at least seven months or longer. In some embodiments, the synovial fluid concentration of the corticosteroid is maintained for a duration of at least eight months or longer. In some embodiments, the synovial fluid concentration of the corticosteroid is maintained for a duration of at least nine months or longer. In some embodiments, the synovial fluid concentration of the corticosteroid is maintained for a duration of at least 10 months or longer. In some embodiments, the synovial fluid concentration of the corticosteroid is maintained for a duration of at least 11 months or longer. In some embodiments, the synovial fluid concentration of the corticosteroid is maintained for a duration of at least 12 months or longer. In some embodiments, the synovial fluid concentration of the corticosteroid is maintained for a duration in the range of about 3 months to at least about 12 months.

In some embodiments, the synovial fluid concentration of the corticosteroid is maintained by administering at least one additional dose of the corticosteroid. In some embodiments, at least one additional dose of the corticosteroid is administered as an extended-release formulation. In some embodiments, at least one additional dose of the corticosteroid is administered as a controlled- or sustained-release formulation. In some embodiments, the corticosteroid is released from the formulation for a duration of at least between 14 days and 90 days. In some embodiments, the corticosteroid is released from the formulation for a duration of at least between 30 days and 90 days. In some embodiments, the corticosteroid is released from the formulation for a duration of at least 3 months. In some embodiments, the corticosteroid is released from the formulation for a duration of at least between 3 months and 12 months. In some embodiments, the corticosteroid is released from the formulation for a duration of at least between 3 months and 6 months. In some embodiments, the corticosteroid is released from the formulation for a duration of at least between 6 months and 12 months.

In some embodiments, the formulation is administered as one or more injections. In some embodiments, the injection is one or more local injections at a site of pain. In some embodiments, the injection is one or more intra-articular or peri-articular injections.

In some embodiments, the disease or disorder associated with joint pain and/or joint inflammation is osteoarthritis, rheumatoid arthritis, acute gouty arthritis, and/or synovitis.

The disclosure also provides methods for maximizing analgesic effect and maintaining maximal analgesic effect over a prolonged duration in a patient with a disease or disorder associated with joint pain and/or joint inflammation—by administering to a subject in need thereof an extended release formulation, e.g., a controlled- or sustained-release formulation, comprising triamcinolone acetonide (TCA), and maintaining a synovial fluid concentration of TCA of at least 6 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of TCA of at least 10 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of TCA of at least 15 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a synovial fluid concentration of TCA of at least 20 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

In some embodiments, the synovial fluid concentration of TCA is maintained at a concentration in the range of about 6 ng/ml to about 1000 ng/ml. In some embodiments, the synovial fluid concentration of TCA is maintained at a concentration in the range of about 10 ng/ml to about 1000 ng/ml. In some embodiments, the synovial fluid concentration of TCA is maintained at a concentration in the range of about 15 ng/ml to about 1000 ng/ml. In some embodiments, the synovial fluid concentration of TCA is maintained at a concentration in the range of about 20 ng/ml to about 1000 ng/ml.

In some embodiments, the synovial fluid concentration of TCA is maintained at a concentration in the range of about 6 ng/ml to about 78 ng/ml. In some embodiments, the synovial fluid concentration of TCA is maintained at a concentration in the range of about 10 ng/ml to about 78 ng/ml. In some embodiments, the synovial fluid concentration of TCA is maintained at a concentration in the range of about 15 ng/ml to about 78 ng/ml. In some embodiments, the synovial fluid concentration of TCA is maintained at a concentration in the range of about 20 ng/ml to about 78 ng/ml.

In some embodiments, the synovial fluid concentration of TCA is maintained for a duration of at least 50 days. In some embodiments, the synovial fluid concentration of TCA is maintained for a duration of at least 75 days. In some embodiments, the synovial fluid concentration of TCA is maintained for a duration of at least 90 days. In some embodiments, the synovial fluid concentration of TCA is maintained for a duration of at least four months or longer. In some embodiments, the synovial fluid concentration of TCA is maintained for a duration of at least five months or longer. In some embodiments, the synovial fluid concentration of TCA is maintained for a duration of at least six months or longer. In some embodiments, the synovial fluid concentration of TCA is maintained for a duration of at least seven months or longer. In some embodiments, the synovial fluid concentration of TCA is maintained for a duration of at least eight months or longer. In some embodiments, the synovial fluid concentration of TCA is maintained for a duration of at least nine months or longer. In some embodiments, the synovial fluid concentration of TCA is maintained for a duration of at least 10 months or longer. In some embodiments, the synovial fluid concentration of TCA is maintained for a duration of at least 11 months or longer. In some embodiments, the synovial fluid concentration of TCA is maintained for a duration of at least 12 months or longer. In some embodiments, the synovial fluid concentration of TCA is maintained for a duration in the range of about 3 months to at least about 12 months.

While suitable formulations of the disclosure include any formulation that achieves and maintains a synovial fluid concentration in the desired range, e.g., a range of at least 6 ng/ml for TCA, formulations that achieve and maintain a synovial fluid concentration significantly higher than the desired level, e.g. above an upper level of around 1000 ng/ml for TCA, will become less effective as a result of unwanted side effects of administering and/or maintaining such high levels of corticosteroid concentrations. For example, it is known that higher systemic concentrations of corticosteroid can lead to suppression of the hypothalamicpituitary-adrenal (HPA) axis, leading to a variety of unwanted side effects.

The examples provided herein use TCA, but this discovery is not limited to TCA or even to Class B corticosteroids in general. The relative potency and protein binding of various corticosteroids are known in the art (See e.g., Derendorf H, Nave R, Drollmann A, Cerasoli F, Wurst W. Relevance of pharmacokinetics and pharmacodynamics of inhaled corticosteroids to asthma. Eur Respir J 2006; 28:1042-1050; Chan W L, Carrell R W, Zhou A, Read R J. How Changes in Affinity of Corticosteroid-binding Globulin Modulate Free Cortisol Concentration. J Clin Endocrinol Metab 2013 August; 98(8):3315-3322; Wilkinson M and Jones B S. Electrophoretic Studies of Synovial Fluid Proteins. Ann Rheum Dis 1964; 23:22).

As such, one of ordinary skill in the art would be able to use the data and information gathered for the post-administration synovial fluid concentration of TCA to calculate and determine the appropriate range of synovial fluid concentration needed to achieve and maintain maximal analgesic effect over a prolonged duration and avoid untoward, undesired, or otherwise deleterious systemic effects in a patient for any given corticosteroid, i.e., to calculate synovial fluid concentrations equivalent to 6 ng/ml TCA and 1000 ng/ml TCA for different corticosteroids. These calculations have been made for exemplary corticosteroids from all classes of corticosteroids, such as, by way of non-limiting example, cortisol, ciclesonide (monopropionate), beclometasone diproprionate, dexamethasone, flunisolide, triamcinolone acetonide, budesonide, desisobutyryl-ciclesonide, fluticasone propionate, and mometasone furoate. These calculations have been made by taking into account relative receptor affinity, relative synovial fluid protein binding, and synovial fluid protein concentrations in osteoarthritis patients.

The range of synovial fluid concentration needed to achieve and maintain maximal analgesic effect over a prolonged duration and avoid untoward, undesired, or otherwise deleterious systemic effects for a variety of exemplary corticosteroids is shown below in Table 1. Suitable synovial concentrations include any intermediate value that falls within the ranges recited below in Table 1.

TABLE 1
Target Synovial Fluid Concentration To Achieve And Maintain Maximal
Analgesic Effect Over A Prolonged Duration And Avoid Untoward,
Undesired, Or Otherwise Deleterious Systemic Effects

| Corticosteroid | Synovial Concentration (ng/ml) |
|---|---|
| cortisol | 144.98 ng/ml-24.16 μg/ml |
| ciclesonide (monopropionate) | 168.93 ng/ml-28.15 μg/ml |
| beclometasone diproprionate | 32.07 ng/ml-5344.96 ng/ml |
| dexamethasone | 14.63 ng/ml-2438.88 ng/ml |
| flunisolide | 8.63 ng/ml-1438.27 ng/ml |
| triamcinolone acetonide (TCA) | 6 ng/ml-1,000 ng/ml |
| budesonide | 1.84 ng/ml-307.11 ng/ml |
| desisobutyryl-ciclesonide | 1.69 ng/ml-281.55 ng/ml |
| fluticasone propionate | 0.95 ng/ml-157.58 ng/ml |
| mometasone furoate | 0.77 ng/ml-128.93 ng/ml |

In some embodiments, the synovial fluid concentration of the corticosteroid is maintained by administering at least one additional dose of the corticosteroid. In some embodiments, at least one additional dose of the corticosteroid is administered as an extended release, e.g., a controlled- or sustained-release formulation. In some embodiments, the corticosteroid is released from the formulation for a duration of at least between 14 days and 90 days. In some embodiments, the corticosteroid is released from the formulation for a duration of at least between 30 days and 90 days. In some embodiments, the corticosteroid is released from the formulation for a duration of at least 3 months. In some embodiments, the corticosteroid is released from the formulation for a duration of at least between 3 months and 12 months. In some embodiments, the corticosteroid is released from the formulation for a duration of at least between 3 months and 6 months. In some embodiments, the corticosteroid is released from the formulation for a duration of at least between 6 months and 12 months.

In some embodiments, the formulation is administered as one or more injections. In some embodiments, the injection is one or more local injections at a site of pain. In some embodiments, the injection is one or more intra-articular or peri-articular injections.

### DEFINITIONS

The following terms are used throughout this disclosure.

“Patient” refers to a human diagnosed with a disease or condition that can be treated in accordance to the inventions described herein. In some embodiments it is contemplated that the formulations described herein may also be used in horses and other animals.

“Delivery” refers to any means used to place the drug into a patient. Such means may include without limitation, placing matrices into a patient that release the drug into a target area. One of ordinary skill in the art recognizes that the matrices may be delivered by a wide variety of methods, e.g., injection by a syringe, placement into a drill site, catheter or cannula assembly, or forceful injection by a gun type apparatus or by placement into a surgical site in a patient during surgery.

The terms “treatment” and “treating” a patient refer to reducing, alleviating, stopping, blocking, delaying the progression of the disease state and/or the symptoms of the disease state, or preventing the symptoms of pain and/or inflammation in a patient. As used herein, “treatment” and “treating” includes partial alleviation of symptoms as well as complete alleviation of the symptoms for a time period. The time period can be hours, days, months, or even years.

By an “effective” amount or a “therapeutically effective amount” of a drug or pharmacologically active agent is meant a nontoxic but sufficient amount of the drug or agent to provide the desired effect, e.g., analgesia. An appropriate “effective” amount in any individual case may be determined by one of ordinary skill in the art using routine experimentation.

“Site of a patient's pain” refers to any area within a body causing pain, e.g., a knee joint with osteoarthritis, nerve root causing sciatic pain, nerve fibers growing into annular tears in discs causing back pain, temporomandibular joint (TMJ) pain, for example TMJ pain associated with temporomandibular joint disorder (TMJD) or pain radiating from epidural or perineural spaces. The pain perceived by the patient may result from inflammatory responses, mechanical stimuli, chemical stimuli, thermal stimuli, as well as allodynia.

Additionally, the site of a patient's pain can comprise one or multiple sites in the spine, such as between the cervical, thoracic, or lumbar vertebrae, or can comprise one or multiple sites located within the immediate area of inflamed or injured joints such as the shoulder, hip, hand, or other joints.

A “biocompatible” material refers to a material that is not toxic to the human body, it is not carcinogenic and it should induce limited or no inflammation in body tissues. A “biodegradable” material refers to a material that is degraded by bodily processes (e.g., enzymatic) to products readily disposable by the body or absorbed into body tissue. The biodegraded products should also be biocompatible with the body. In the context of intra-articular drug delivery systems for TCA and other corticosteroids, such polymers may be used to fabricate, without limitation: microparticles, micro-spheres, matrices, microparticle matrices, micro-sphere matrices, capsules, hydrogels, rods, wafers, pills, liposomes, fibers, pellets, or other appropriate pharmaceutical delivery compositions that a physician can administer into the joint. The biodegradable polymers degrade into non-toxic residues that the body easily removes or break down or dissolve slowly and are cleared from the body intact. The polymers may be cured ex-vivo forming a solid matrix that incorporates the drug for controlled release to an inflammatory region. Suitable biodegradable polymers may include, without limitation natural or synthetic biocompatible biodegradable material.

Descriptions of various embodiments of the invention are given below. Although these embodiments are exemplified with reference to treat joint pain associated with osteoarthritis, rheumatoid arthritis and other joint disorders, it should not be inferred that the invention is only for these uses. Rather, it is contemplated that embodiments of the present invention will be useful for treating other forms of joint pain by administration into articular and peri-articular spaces. In addition, it will be understood that for some embodiments injection near a joint may be equivalent to injections in that joint. Any and all uses of specific words and references are simply to detail different embodiments of the present invention.

Local administration of a corticosteroid formulation, e.g., a TCA formulation, can occur, for example, by injection into the intra-articular space or peri-articular space at or near the site of a patient's pain and/or structural tissue damage. Local injection of the formulations described herein into articular or peri-articular spaces may be useful in the treatment of, for example, juvenile rheumatoid arthritis, sciatica and other forms of radicular pain (e.g., arm, neck, lumbar, thorax), psoriatic arthritis, acute gouty arthritis, Morton's neuroma, acute and subacute bursitis, acute and subacute nonspecific tenosynovitis and epicondylitis, and ankylosing spondylitis.

In one embodiment, the corticosteroid formulations, e.g., TCA formulations, provided herein are useful in treating, alleviating a symptom of, ameliorating and/or delaying the progression of sciatica. In one embodiment, corticosteroid formulations, e.g., TCA formulations, provided herein are useful in treating, alleviating a symptom of, ameliorating and/or delaying the progression of temporomandibular joint disorder (TMJD).

Administration of a corticosteroid formulation, e.g., TCA formulation, to a patient suffering from an inflammatory disease such as osteoarthritis or rheumatoid arthritis, is considered successful if any of a variety of laboratory or clinical results is achieved. For example, administration of a corticosteroid formulation, e.g., TCA formulation is considered successful if one or more of the symptoms associated with the disease is alleviated, reduced, inhibited or does not progress to a further, i.e., worse, state. Administration of a corticosteroid formulation, e.g., TCA formulation is considered successful if the disease, e.g., an arthritic or other inflammatory disease, or any symptom thereof enters remission or does not progress to a further, i.e., worse, state.

Also, any and all alterations and further modifications of the invention, as would occur to one of ordinary skill in the art, are intended to be within the scope of the invention.

### Corticosteroids:

The Examples provided herein demonstrate formulations of TCA for achieving and maintaining maximal analgesic effect over a prolonged duration following intra-articular administration. However, these examples are not limiting to the scope of the disclosure, and the skilled artisan would appreciate that other corticosteroid formulations are also useful for achieving and maintaining maximal analgesic effect over a prolonged duration following intra-articular administration.

Corticosteroids associated with embodiments of the present invention can be any naturally occurring or synthetic steroid hormone. Naturally occurring corticosteroids are secreted by the adrenal cortex or generally the human body.

Corticosteroid molecules have the following basic structure:

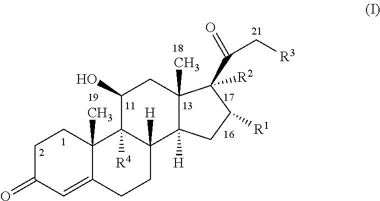

Corticosteroids have been classified into four different groups (A, B, C, and D). (See e.g., Foti et al. “Contact Allergy to Topical Corticosteroids: Update and Review on Cross-Sensitization.” Recent Patents on Inflammation & Allergy Drug Discovery 3 (2009): 33-39; Coopman et al., “Identification of cross-reaction patterns in allergic contact dermatitis to topical corticosteroids.” Br J Dermatol 121 (1989): 27-34). Class A corticosteroids are hydrocortisone types with no modification of the D ring or C20-C21 or short chain esters on C20-C21. Main examples of Class A corticosteroids include prednisolone, hydrocortisone and methylprednisolone and their ester acetate, sodium phosphate and succinate, cortisone, prednisone, and tixocortol pivalate. Class B corticosteroids are triamcinolone acetonide (TCA) types with cis/ketalic or diolic modifications on C16-C17. Main examples of Class B corticosteroids include triamcinolone acetonide (TCA), triamcinolone hexacetonide, fluocinolone acetonide, amcinonide, desonide, fluocinonide, halcinonide, budesonide, and flunisolide. Class C corticosteroids are betamethasone types with a —CH3 modification on C16, but no esterification on C17-C21. Main examples of Class C corticosteroids include betamethasone, dexamethasone, desoxymethasone, fluocortolone, and halomethasone. Class D corticosteroids are clobetasone or hydrocortisone esterified types with a long chain on C17 and/or C21 and with no methyl group on C16. Main examples of Class D corticosteroids include fluticasone, clobetasone butyrate, clobetasol propionate, hydrocortisone-17-aceponate, hydrocortisone-17-butyrate, beclomethasone dipropionate, betamethasone-17-valerate, betamethasone dipropionate, methylprednisolone aceponate, and prednicarbate.

For the present invention non-limiting examples of corticosteroids may include: betamethasone, betamethasone acetate, betamethasone dipropionate, betamethasone 17-valerate, cortivazol, dexamethasone, dexamethasone acetate, dexamethasone sodium phosphate, hydrocortisone, hydrocortisone aceponate, hydrocortisone acetate, hydrocortisone butyrate, hydrocortisone cypionate, hydrocortisone probutate, hydrocortisone sodium phosphate, hydrocortisone sodium succinate, hydrocortisone valerate, methylprednisolone, methylprednisolone aceponate, methylprednisolone acetate, methylprednisolone sodium succinate, prednisolone, prednisolone acetate, prednisolone metasulphobenzoate, prednisolone sodium phosphate, prednisolone steaglate, prednisolone tebutate, triamcinolone, triamcinolone acetonide, triamcinolone acetonide 21-palmitate, triamcinolone benetonide, triamcinolone diacetate, triamcinolone hexacetonide, alclometasone, alclometasone dipropionate, amcinonide, amelometasone, beclomethasone, beclomethasone dipropionate, beclomethasone dipropionate monohydrate, budesonide, butixocort, butixocort propionate, ciclesonide, ciprocinonide, clobetasol, clobetasol propionate, clocortolone, clobetasone, clobetasone butyrate, clocortolone pivalate, cloprednol, cortisone, cortisone acetate, deflazacort, domoprednate, deprodone, deprodone propionate, desonide, desoximethasone, desoxycortone, desoxycortone acetate, dichlorisone, diflorasone, diflorasone diacetate, diflucortolone, difluprednate, fluclorolone, fluclorolone acetonide, fludrocortisone, fludrocortisone acetate, fludroxycortide, flumethasone, flumethasone pivalate, flunisolide, fluocinolone, fluocinolone acetonide, fluocortin, fluocortolone, fluorometholone, fluticasone, fluticasone furoate, fluticasone propionate, fluorometholone acetate, fluoxymesterone, fluperolone, fluprednidene, fluprednidene acetate, fluprednisolone, formocortal, halcinonide, halobetasol propionate, halometasone, halopredone, halopredone acetate, hydrocortamate, isoflupredone, isoflupredone acetate, itrocinonide, loteprednol etabonate, mazipredone, meclorisone, meclorisone dibutyrate, medrysone, meprednisone, mometasone, mometasone furoate, mometasone furoate monohydrate, nivacortol, paramethasone, paramethasone acetate, prednazoline, prednicarbate, prednisolone, prednylidene, procinonide, rofleponide, rimexolone, timobesone, tipredane, tixocortol, tixocortol pivalate and tralonide.

### TCA:

Triamcinolone acetonide (TCA) associated with embodiments of the present invention has the following basic structure:

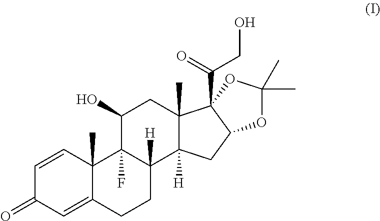

For the present invention non-limiting examples of TCA may include triamcinolone acetonide and/or pharmaceutically acceptable salts thereof.

Embodiments of the invention include using extended release, e.g., sustained release, TCA formulations delivered to treat pain at dosages that do not adversely suppress the HPA axis. Such amounts delivered locally to relieve pain due to inflammation, will provide a systemic concentration that does not have a measurable adverse effect on the HPA axis (differences if any are not significant because any such differences are within normal assay variability) or, as desired, may have a measurable but clinically insignificant effect on the HPA axis (basal cortisol is suppressed to some measurable extent but stress responses are adequately preserved). Further embodiments of the invention may include doses during a second period of time selected to adjust for a change in sensitivity of the HPA axis to suppression following exposure during a first period of time to TCA.

In some embodiments, a single component TCA extended release formulation, e.g., sustained release formulation, releases a TCA dose (e.g., in mg/day) that suppresses the HPA axis by no more than between 5-40% at steady state, more preferably no more than between 10-35% at steady state. These doses are therapeutically effective without adverse side effects.

In some embodiments, a single component TCA extended release formulation, e.g., sustained release formulation, releases a dose (e.g., in mg/day) that does not measurably suppress the HPA axis at steady state. These doses are therapeutically effective without adverse side effects.

### Extended Release Delivery Platforms, Controlled or Sustained Release Delivery Platforms:

The disclosure encompasses any extended release corticosteroid formulation, e.g., any controlled- or sustained-release corticosteroid formulation, that produces and maintains maximal analgesic effect in a patient for an extended period of time post administration. Suitable formulations can vary in composition, components, dosing, etc., provided that the corticosteroid formulation is effective to deliver the corticosteroid over an extended period of time and to maintain a synovial fluid concentration of corticosteroid in the desired range.

In some embodiments, the corticosteroid formulation is a prodrug or prodrug-based formulation in which an unmodified active agent, e.g., a corticosteroid, is released at predetermined rates governed by variables such as physiological pH and temperature condition. For example, the formulation includes an inactive prodrug that includes at least the active agent and a carrier. In some embodiments, the formulation includes a linker.

In some embodiments, the extended release formulation, e.g., the sustained release formulation, includes a matrix, such as for example, a hydrogel-based matrix, a hyaluronic acid-based matrix, and/or a biodegradable polymer-based matrix. In some embodiments, the hydrogel is a polyurethane hydrogel, a polyacrylate hydrogel, a gelatin hydrogel, a carboxymethyl cellulose hydrogel, a pectin hydrogel, an alginate hydrogel, and/or a hyaluronic acid hydrogel. In some embodiments, the biodegradable polymer is selected from, but not limited to, PLGA, PLA, PGA, polycaprolactone, polyhydroxybutyrate, polyorthoesters, polyalkaneanhydrides, gelatin, collagen, oxidized cellulose, and/or polyphosphazene.

In some embodiments, the extended release formulation, e.g., the sustained release formulation, includes a biodegradable polymer. In some embodiments, the extended release formulation, e.g., the sustained release formulation, includes a biodegradable polymer microparticle formulation. The manufacture of extended release microparticles, e.g., sustained-release microparticles, or methods of making biodegradable polymer microparticles are known in the art. In some embodiments, the extended release formulation, e.g., the sustained-release microparticle or other sustained-release formulation, is PLGA-based. PLGA microparticles are commercially available from a number of sources and/or can be made by, but not limited to, spray drying, solvent evaporation, phase separation, fluidized bed coating or combinations thereof.

If not purchased from a supplier, then the biodegradable PLGA copolymers may be prepared by the procedure set forth in U.S. Pat. No. 4,293,539 (Ludwig, et al.), the disclosure of which is hereby incorporated by reference in its entirety. Ludwig prepares such copolymers by condensation of lactic acid and glycolic acid in the presence of a readily removable polymerization catalyst (e.g., a strong acid ion-exchange resin such as Dowex HCR-W2-H). However, any suitable method known in the art of making the polymer can be used.

In the coacervation process, a suitable biodegradable polymer is dissolved in an organic solvent. Suitable organic solvents for the polymeric materials include, but are not limited to acetone, halogenated hydrocarbons such as chloroform and methylene chloride, aromatic hydrocarbons such as toluene, halogenated aromatic hydrocarbons such as chlorobenzene, and cyclic ethers such as dioxane. The organic solvent containing a suitable biodegradable polymer is then mixed with a non-solvent or anti-solvent such as silicone based solvent. By mixing the miscible non-solvent in the organic solvent, the polymer precipitates out of solution in the form of liquid droplets. The liquid droplets are then mixed with another non-solvent, such as heptane or petroleum ether, to form the hardened microparticles. The microparticles are then collected and dried. Process parameters such as solvent and non-solvent selections, polymer/solvent ratio, temperatures, stirring speed and drying cycles are adjusted to achieve the desired particle size, surface smoothness, and narrow particle size distribution.

In the phase separation or phase inversion procedures entrap dispersed agents in the polymer to prepare microparticles. Phase separation is similar to coacervation of a biodegradable polymer. By addition of a non-solvent such as petroleum ether, to the organic solvent containing a suitable biodegradable polymer, the polymer is precipitated from the organic solvent to form microparticles.

In the salting out process, a suitable biodegradable polymer is dissolved in an aqueous miscible organic solvent. Suitable water miscible organic solvents for the polymeric materials include, but are not limited to acetone, acetonitrile, and tetrahydrofuran. The water miscible organic solvent containing a suitable biodegradable polymer is then mixed with an aqueous solution containing salt. Suitable salts include, but are not limited to electrolytes such as magnesium chloride, calcium chloride, or magnesium acetate and non-electrolytes such as sucrose. The polymer precipitates from the organic solvent to form microparticles, which are collected and dried. Process parameters such as solvent and salt selection, polymer/solvent ratio, temperatures, stirring speed and drying cycles are adjusted to achieve the desired particle size, surface smoothness, and narrow particle size distribution.

Alternatively, the microparticles may be prepared by the process of Ramstack et al., 1995, described in published international patent application WO 95/13799, the disclosure of which is incorporated herein in its entirety. The Ramstack et al. process essentially provides for a first phase, including an active agent and a polymer, and a second phase, that are pumped through a static mixer into a quench liquid to form microparticles containing the active agent. The first and second phases can optionally be substantially immiscible and the second phase is preferably free from solvents for the polymer and the active agent and includes an aqueous solution of an emulsifier.

In the spray drying process, a suitable biodegradable polymer is dissolved in a suitable solvent and then sprayed through nozzles into a drying environment provided with sufficient elevated temperature and/or flowing air to effectively extract the solvent.

Alternatively, a suitable biodegradable polymer can be dissolved or dispersed in supercritical fluid, such as carbon dioxide. The polymer is either dissolved in a suitable organic solvent, such as methylene chloride, prior to mixing in a suitable supercritical fluid or directly mixed in the supercritical fluid and then sprayed through a nozzle. Process parameters such as spray rate, nozzle diameter, polymer/solvent ratio, and temperatures, are adjusted to achieve the desired particle size, surface smoothness, and narrow particle size distribution.

In a fluidized bed coating, the drug is dissolved in an organic solvent along with the polymer. The solution is then processed, e.g., through a Wurster air suspension coating apparatus to form the final microcapsule product. In some embodiments, the microcapsule product is formed using a spinning disk methodology, e.g., a Southwest Research Institute (SwRI) technology.

The microparticles can be prepared in a size distribution range suitable for local infiltration or injection. The diameter and shape of the microparticles can be manipulated to modify the release characteristics. In addition, other particle shapes, such as, for example, cylindrical shapes, can also modify release rates of an extended release TCA formulation, e.g., a sustained release TCA formulation, by virtue of the increased ratio of surface area to mass inherent to such alternative geometrical shapes, relative to a spherical shape. The microparticles have a volumetric mean diameter ranging between about 0.5 to 500 microns. In some embodiments, the microparticles have a volumetric mean diameter of between 10 to about 100 microns.

Biodegradable polymer microparticles that deliver extended release TCA formulations, e.g., sustained release TCA formulations, may be suspended in suitable aqueous or non-aqueous carriers which may include, but are not limited to water, saline, pharmaceutically acceptable oils, low melting waxes, fats, lipids, liposomes and any other pharmaceutically acceptable substance that is lipophilic, substantially insoluble in water, and is biodegradable and/or eliminable by natural processes of a patient's body. Oils of plants such as vegetables and seeds are included. Examples include oils made from corn, sesame, canola, soybean, castor, peanut, olive, arachis, maize, almond, flax, safflower, sunflower, rape, coconut, palm, babassu, and cottonseed oil; waxes such as carnoba wax, beeswax, and tallow; fats such as triglycerides, lipids such as fatty acids and esters, and liposomes such as red cell ghosts and phospholipid layers.

### Excipients:

The release rate of a corticosteroid, e.g., TCA, from a formulation can be modulated or stabilized by adding one or more pharmaceutically acceptable excipient(s) to the formulation. In some embodiments, additional excipient(s) may include any useful ingredient added to the biodegradable polymer depot that is not a corticosteroid or a biodegradable polymer. In some embodiments, additional excipient(s) may include a mixture of multiple polymers added to the biodegradable polymer depot to adjust the release profile as necessary. Pharmaceutically acceptable excipients may include without limitation lactose, dextrose, sucrose, sorbitol, mannitol, starches, gum acacia, calcium phosphate, alginates, tragacanth, gelatin, calcium silicate, microcrystalline cellulose, PEG, polysorbate 20, polysorbate 80, polyvinylpyrrolidone, cellulose, water, saline, syrup, methyl cellulose, and carboxymethyl cellulose. An excipient for modulating the release rate of corticosteroid, e.g., TCA, from the formulation may also include without limitation pore formers, pH modifiers, solubility enhancers, reducing agents, antioxidants, and free radical scavengers.

### Delivery of Corticosteroid Formulations:

Parenteral administration of corticosteroid, e.g., TCA, formulations of the invention can be effected by intra-articular injection or other injection using a needle. To inject the corticosteroid, e.g., TCA, formulations into a joint, needles having a gauge of about 14-28 gauge are suitable. It will be appreciated by those skilled in the art that corticosteroid, e.g., TCA, formulations of the present invention may be delivered to a treatment site by other conventional methods, including catheters, infusion pumps, pens devices, injection guns and the like.

### Indications

Descriptions of various embodiments of the invention are given below. Although these embodiments are exemplified with reference to treat joint pain associated with osteoarthritis, rheumatoid arthritis and other joint disorders, it should not be inferred that the invention is only for these uses. Rather, it is contemplated that embodiments of the present invention will be useful for treating other forms of joint pain by administration into articular and peri-articular spaces. In addition, it will be understood that for some embodiments injection near a joint may be equivalent to injections in that joint. It is also contemplated that embodiments of the present invention may be useful for injection or administration into soft tissues or lesions. Any and all uses of specific words and references are simply to detail different embodiments of the present invention.

Local administration of a corticosteroid microparticle formulation can occur, for example, by injection into the intra-articular space, peri-articular space, soft tissues, lesions, epidural space, perineural space, or the foramenal space at or near the site of a patient's pain and/or structural tissue damage. Local injection of the formulations described herein into articular or peri-articular spaces may be useful in the treatment of, for example, juvenile rheumatoid arthritis, sciatica and other forms of radicular pain (e.g., arm, neck, lumbar, thorax), psoriatic arthritis, acute gouty arthritis, Morton's neuroma, acute and subacute bursitis, acute and subacute nonspecific tenosynovitis and epicondylitis, acute rheumatic carditis and ankylosing spondylitis. Injection of the microparticles described herein into soft tissues or lesions may be useful in the treatment of, for example, alopecia areata, discoid lupus, erythematosus; keloids, localized hypertrophic, infiltrated inflammatory lesions of granuloma annulare, lichen planus, lichen simplex chronicus (neurodermatitis), psoriasis and psoriatic plaques; necrobiosis lipoidica diabeticorum, and psoriatic arthritis. Injection of the microparticles described herein into epidural spaces may be useful in the treatment of, for example, Neurogenic Claudication (NC). Intramuscular or other soft tissues or lesions injections may also be useful in providing systemic exposures that are effective in the control of incapacitating allergic conditions (including but not limited to asthma, atopic dermatitis, contact dermatitis, drug hypersensitivity reactions, seasonal or perennial allergic rhinitis, serum sickness, transfusion reactions), bullous dermatitis herpetiformis, exfoliative dermatitis, mycosis fungoides, pemphigus, severe erythema multiforme (Stevens-Johnson syndrome), Primary or secondary adrenocortical insufficiency in conjunction with mineralocorticoids where applicable; congenital adrenal hyperplasia, hypercalcemia associated with cancer, nonsupportive thyroiditis, exacerbations of regional enteritis and ulcerative colitis, acquired (autoimmune) hemolytic anemia, congenital (erythroid) hypoplastic anemia (Diamond blackfan anemia), pure red cell aplasia, select cases of secondary thrombocytopenia, trichinosis with neurologic or myocardial involvement, tuberculous meningitis with subarachnoid block or impending block when used concurrently with appropriate antituberculous chemotherapy, palliative management of leukemias and lymphomas, acute exacerbations of multiple sclerosis, cerebral edema associated with primary or metastatic brain tumor or craniotomy, to induce diuresis or remission of proteinuria in idiopathic nephrotic syndrome, or to induce diuresis or remission of proteinuria in lupus erythematosus, berylliosis, symptomatic sarcoidosis, fulminating or disseminated pulmonary tuberculosis (when used concurrently with appropriate antituberculous chemotherapy), idiopathic eosinophilic pneumonias, symptomatic sarcoidosis, dermatomyositis, polymyositis, and systemic lupus erythematosus, post-operative pain and swelling.

In one embodiment, the corticosteroid microparticle formulations provided herein are useful in treating, alleviating a symptom of, ameliorating and/or delaying the progression of sciatica. In one embodiment, corticosteroid microparticle formulations provided herein are useful in treating, alleviating a symptom of, ameliorating and/or delaying the progression of temporomandibular joint disorder (TMJD).

In one embodiment, the corticosteroid microparticle formulations provided herein are useful in treating, alleviating a symptom of, ameliorating and/or delaying the progression of Neurogenic Claudication (NC) secondary to lumbar spinal stenosis (LSS). LSS implies spinal canal narrowing with possible subsequent neural compression (classified by anatomy or etiology). Neurogenic Claudication (NC) is a hallmark symptom of lumbar stenosis, in which the column of the spinal cord (or the canals that protect the nerve roots) narrows at the lower back. This narrowing can also occur in the spaces between the vertebrae where the nerves leave the spine to travel to other parts of the body.

The microparticles of the invention are used to treat, alleviate a symptom of, ameliorate and/or delay the progression in patients suffering from NC secondary to LSS. The corticosteroid microparticle formulations can be administered, for example, by epidural steroid injection (ESI).

Administration of a corticosteroid microparticle formulation, e.g., a TCA microparticle formulation, to a patient suffering from an inflammatory disease such as osteoarthritis or rheumatoid arthritis, is considered successful if any of a variety of laboratory or clinical results is achieved. For example, administration of a corticosteroid microparticle formulation is considered successful if one or more of the symptoms associated with the disease is alleviated, reduced, inhibited or does not progress to a further, i.e., worse, state. Administration of a corticosteroid microparticle formulation is considered successful if the disease, e.g., an arthritic or other inflammatory disease, enters remission or does not progress to a further, i.e., worse, state.

Also, any and all alterations and further modifications of the invention, as would occur to one of ordinary skill in the art, are intended to be within the scope of the invention

All references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: Materials and Methods

### Trials Design

Two sequential studies are described, The initial 6-Week Study was a double-blind, randomized, parallel-group, active comparator study in patients with OA of the knee following injection of 10, 40, or 60 mg of TCA Formulation 1 or 40 mg of TCA IR. The follow-on 20-Week Study was an open-label study in patients with OA of the knee following a single IA injection of 10 or 40 mg of TCA Formulation 1, or 40 mg of TCA IR.

Unless otherwise specified, the design elements of the 6- and 20-Week Studies were the same.

Participants:

Eligible patients gave informed consent to participate in the study and were at least 35 years old (at least 40 years old in the 20-Week Study), with a body mass index ≦40 kg/m² and a diagnosis of OA of the knee for at least 6 months prior to screening consistent with the clinical and radiological criteria of the American College of Rheumatology Criteria. In the 6-Week Study, morning serum cortisol results were within normal range at screening. Patients agreed to abstain from using restricted medications during the study. Patients were excluded if they had received an IA corticosteroid in any joint within 3 months of screening; oral, inhaled, or intranasal corticosteroids within 1 month; or IA hyaluronic acid in the index knee within 6 months.

Interventions:

Patients received a single IA injection of TCA Formulation 1 at 10, or 40 mg or TCA IR 40 mg on Day 1 (a 60 mg TCA Formulation 1 arm was also included in the 6-Week Study). Prior to injection, the index knee was cleansed and sprayed with ethyl chloride. Following aspiration of synovial fluid, 3 ml of the reconstituted TCA Formulation 1 or 1 ml of TCA IR was injected into the synovial space using a 22-gauge, 1.5- or 2-inch needle. In patients with bilateral OA, the knee with greater baseline pain was injected.

Bioanalytical Method:

Human Plasma samples were assayed for TCA concentrations using a validated High Performance Liquid Chromatographic Method with Tandem Mass Spectrometry Detection. The method used an internal standard (triamcinolone-6-d1 acetonide-d6), and both TCA and internal standard were extracted and separated using an isocratic chromatographic separation using a C18 column solid phase and a water/methanol mobile phase. Detection was by tandem mass spectrometry. The validated range was from 50.06 to 5006.00 pg/ml and calibration standards for sample assay ranged from 50.00 to 5000.00 pg/ml.

Human Synovial Fluid samples were assayed for TCA concentrations using a validated High Performance Liquid Chromatographic Method with Tandem Mass Spectrometry Detection. The method used an internal standard (triamcinolone-6-d1 acetonide-d6), and both TCA and internal standard were extracted with an automated liquid-liquid extraction using a mixture of methyl tertbutyl ether and hexanes. Detection was by tandem mass spectrometry. The validated range was from 50.00 to 50000.00 pg/ml and calibration standards for sample assay ranged from 50.00 to 50000.00 pg/ml.

Table 2 shows baseline data. In the TCA IR group, 3 patients had synovial fluid available for analysis at Week 6; 5 patients had synovial fluid available for analysis at Week 12. In the TCA Formulation 1 10 mg group, 5 patients had synovial fluid available for analysis at Week 6; 8 patients had synovial fluid available for analysis at Week 12. In the TCA Formulation 1 40 mg group, 5 patients had synovial fluid available for analysis at Week 6; 6 patients had synovial fluid available for analysis at Week 12; 8 patients at Week 16; and 11 patients at Week 20.

TABLE 2
TCA Formulation 1 and TCA IR, Plasma Pharmacokinetic
Parameters of TCA over six weeks following a single IA
injection of 10 mg TCA Formulation 1, 40 mg TCA Formulation
1, 60 mg TCA Formulation 1 and 40 mg TCA IR.

| | Geometric Mean | | | |
|---|---|---|---|---|
| | Form. 1 | Form. 1 | Form. 1 | TCA IR |
| | 10 mg | 40 mg | 60 mg | 40 mg |
| | N = 5 | N = 7 | N = 7 | N = 5 |
| AUC₀₋ₜ | 77.74 | 335.63 | 630.35 | 767.78 |
| ng · h/ml | | | | |
| (Log SD) | (0.62) | (0.68) | (0.53) | (0.50) |
| AUC_{0-inf} | 125.63 | 416.54 | 734.67 | 877.80 |
| ng · h/ml | | | | |
| (Log SD) | (0.42) | (0.62) | (0.50) | (0.34) |
| AUC₀₋₂₄ | 5.64 | 17.56 | 28.57 | 252.04 |
| ng · h/mL | | | | |
| (Log SD) | (0.41) | (0.73) | (0.66) | (1.66) |
| CL/F ml/h | 79597 | 96030 | 81670 | 45568 |
| (ml/h) | (0.42) | (0.62) | (0.50) | (0.34) |
| Cₘₐₓ ng/ml | 0.31 | 0.88 | 1.50 | 17.54 |
| (Log SD) | (0.34) | (0.71) | (0.64) | (1.65) |
| t_{1/2 H} | 281 | 357 | 354 | 150 |
| (h) | (0.54) | (0.29) | (0.24) | (1.75) |
| tₘₐₓ^{a} | 94.9 | 4.00 | 8.02 | 3.97 |
| (h min, max) | (6.00, 95.4) | (2.00, 6.00) | (2.00, 94.4) | (1.00, 8.00) |
| MRT_{inf} | 395 | 554 | 498 | 116 |
| (h) | (0.43) | (0.25) | (0.21) | (1.70) |

Plasma Pharmacokinetic Parameters over Six Weeks Post Injection: Plasma pharmacokinetic parameters are presented in Table 2. TCA exposure (AUC and Cₘₐₓ) associated with TCA Formulation 1 increased in a proportional manner with dose, as indicated by the similar CL/F observed across dose levels (79597 to 96030 ml/h). In contrast, the CL/F of 40 mg TCA IR was 45568 ml/h. Assuming that the elimination of TCA in the bloodstream is independent of the formulation, the difference between the CL/F of the TCA IR and TCA Formulation 1 may be attributed to a lower bioavailability (F %) of TCA for TCA Formulation 1. The relative F % between TCA IR and TCA Formulation 1 at the matching 40 mg dose level was approximately 50% (473116/919128 pg·h/ml). The lower bioavailability of TCA for TCA Formulation 1 is associated with lower systemic exposure to TCA, and is consistent with the slow release of TCA from TCA Formulation 1 in the synovial tissues.

Systemic and Synovial Fluid TCA Concentration Profiles Over 12 Weeks Post Injection:

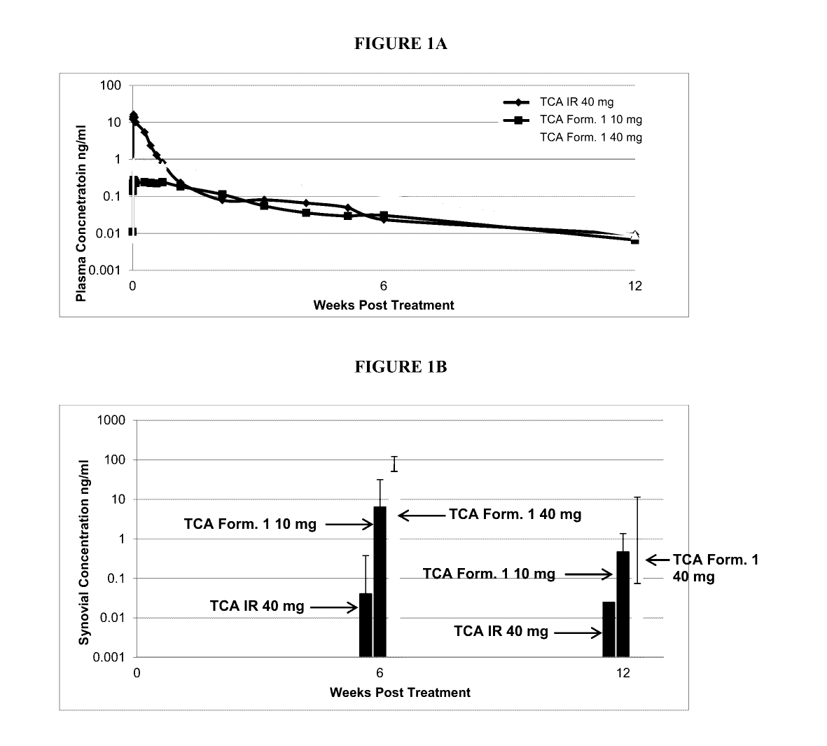

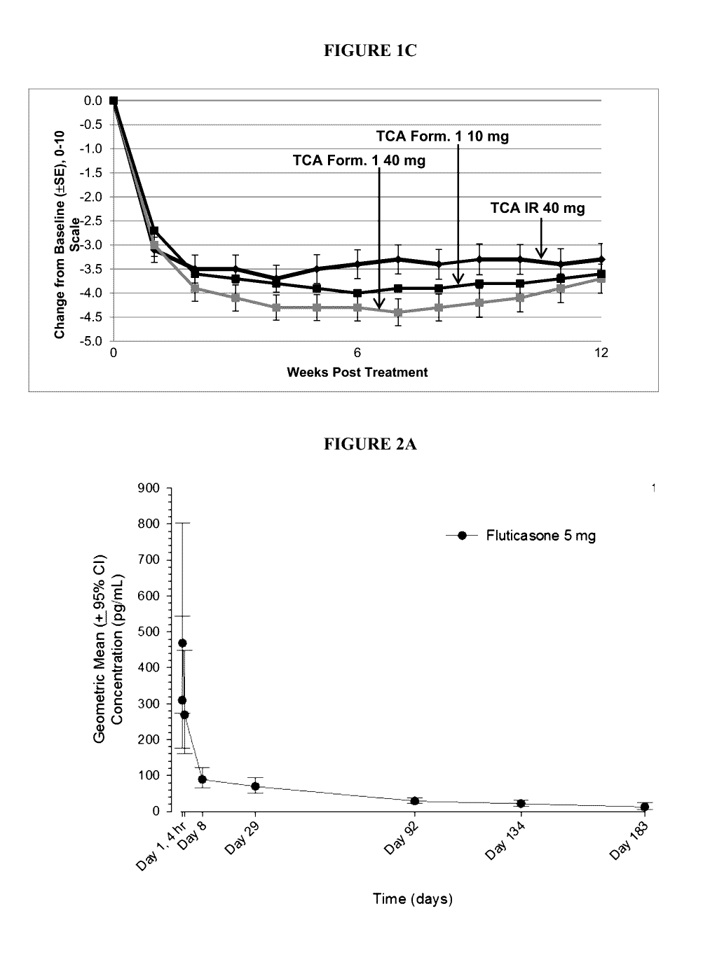

The mean plasma concentration-time profiles over 12 weeks following the administration of 10 and 40 mg TCA Formulation 1 and 40 mg of TCA IR are summarized in FIG. 1A. The plasma profile of TCA IR was characterized by very rapid absorption of TCA into the systemic circulation with peak plasma levels occurring in the first 4 hours post-injection. The decline following peak was rapid and multi-exponential. At the 10 and 40 mg doses, TCA Formulation 1 displayed a relatively slow absorption into the systemic circulation. Peak concentrations for the 40 mg dose of TCA Formulation 1 also occurred within 4 hours; however, these levels were substantially lower than TCA IR (Cₘₐₓ approximately 20× lower than the equivalent dose of TCA IR), and the decline following peak was slow and mono-exponential. Plasma levels associated with 40 mg TCA IR, 10 mg of TCA Formulation 1, and 40 mg of TCA Formulation 1 dropped below LLQ at Week 5, Week 4, and Week 12, respectively. Overall, variability in TCA concentrations was slightly higher in TCA IR as compared to TCA Formulation 1.

The synovial fluid concentration of TCA in patients receiving 40 mg of TCA IR was below the lower limit of quantitation (<0.05 ng/ml) at Week 6 and Week 12. The geometric mean of the synovial fluid concentrations of TCA produced by 10 mg TCA Formulation 1 at Week 6 was 6.48 ng/ml; 95% CI 13.24, 3.17, and at Week 12 was 0.47 ng/ml; 95% CI 0.17, 1.40. The geometric mean of concentrations of synovial fluid concentrations of TCA produced by 40 mg TCA Formulation 1 at Week 6 was 78.75 ng/ml; 95% CI 50.93, 121.77, and at Week 12 was 0.92 ng/ml; 95% CI 0.74, 1.15 (FIG. 1B).

At time points beyond Week 12, measurable concentrations of TCA were observed at Week 16 for the 40 mg TCA Formulation 1, 0.22 ng/ml; 95% CI 0.37, 0.11. At Week 20, synovial fluid levels associated with 40 mg TCA Formulation 1 were below LLQ.

### Example 2. Measuring Synovial Fluid Concentrations of TCA

In two studies of patients with Osteoarthritis (OA) of the knee, plasma pharmacokinetics, synovial fluid concentrations of TCA, and effects on cortisol suppression were evaluated following IA injection of the TCA/PLGA microparticle formulation referred to herein as TCA Formulation 1 and standard, non-extended release TCA suspension (referred to herein as “TCA IR”) at doses known to have analgesic effect.

Plasma concentrations of 40 mg TCA IR dose peaked at 17.54 ng/ml 4 hours post-injection and were undetectable at Weeks 6 and 12; the 40 mg dose of TCA Formulation 1 produced peak concentration of 0.88 ng/ml at 4 hours; 0.11 ng/ml at Week 6 and 0.02 ng/ml at Week 12.

Synovial fluid concentrations of TCA in patients receiving 40 mg of TCA IR were below the lower Level of Quantitation (LLQ) at Weeks 6 and 12. Synovial fluid concentrations of TCA produced by 40 mg of TCA Formulation 1 were 78.75 ng/ml at Week 6 and 0.92 ng/ml at Week 12.

Significantly less suppression of morning serum cortisol was observed with 40 mg TCA Formulation 1 than 40 mg TCA IR at Days 2 and 3 (p=0.0141 and p=0.0027, respectively).

A companion study demonstrated that 40 mg TCA Formulation 1 amplified and prolonged analgesic effect relative to TCA IR. Together, the studies presented herein demonstrate that sustained synovial fluid concentrations of approximately 40 ng/ml are required to optimize the analgesic effect in patients. The corresponding plasma concentrations are unlikely to compromise the stress response in patients with intact HPA axis function.

These studies demonstrate the feasibility of measuring plasma and synovial fluid concentrations of TCA at various time points post-administration. These two studies were the first to report synovial fluid concentrations of a corticosteroid following IA injection.

Systemic Pharmacokinetics:

These studies, overall, demonstrated that the PK profile of TCA Formulation 1 differed substantially from TCA IR. The early peak in systemic concentration of TCA produced by TCA IR was not evident with TCA Formulation 1. Mean Cₘₐₓ for TCA in plasma following injection of the 40 mg dose of TCA Formulation 1 was 20-fold less than that produced by the matching 40 mg dose of TCA IR. The absence of this early peak accounts for the substantial reduction in % Fluctuation with TCA Formulation 1 relative to TCA IR, as all doses of TCA Formulation 1 maintain relatively constant plateau concentrations of TCA in plasma over 12 weeks. The overall systemic AUC of TCA Formulation 1 was lower than that produced by 40 mg TCA IR (approximately a 2× reduction at 40 mg TCA Formulation 1 relative to 40 mg TCA IR). These results suggest that relative to TCA IR, TCA Formulation 1 was associated with the slow release of TCA from the synovial tissues, and elimination of early peak concentrations of TCA may confer safety advantages in patients with compromised glycemic control. (Habib G S, Bashir M, Jabbour A. Increased blood glucose levels following intra-articular injection of methylprednisolone acetate in patients with controlled diabetes and symptomatic osteoarthritis of the knee. Ann Rheum Dis 2008; 67(12):1790-1).

Synovial Fluid Pharmacokinetics:

Relationship to Efficacy: To characterize the relationship between synovial fluid concentrations of TCA and analgesic effect, the synovial fluid concentration data from these two studies were correlated to efficacy assessments from a companion study of safety and efficacy. (Atuorala I, Kwoh C K, Guermazi A, Roemer F W, Boudreau R M, Hannon M J et al.-Synovitis in knee osteoarthritis: a precursor of disease? Ann Rheum Dis 2014; 0:1-6). In the Phase 2 double-blind study, 228 patients with moderate to severe knee OA pain were randomized to an IA injection of TCA Formulation 1 (containing 10, 40, or 60 mg TCA) or 40 mg of TCA IR. Average Daily Pain (ADP) on the 11-point Numeric Rating Scale (NRS) was collected over 12 weeks. The 40 mg dose of TCA Formulation 1 produced pain relief superior to TCA IR at Weeks 5-10, and between Weeks 2 and 12, the magnitude of the analgesic effect of the 40 mg dose of TCA Formulation 1 exceeded the maximum observed effect of 40 mg TCA IR at Week 4 (FIG. 1C). Of note, the acute effect of TCA IR is among the largest reported analgesic effects in OA of the knee (Bjordal J M, Johnson M I, Lopes-Martins R A B, Bogen B, Chow R, Ljunggren A E. Short-term efficacy of physical interventions in osteoarthritic knee pain. A systematic review and meta-analysis of randomised placebo-controlled trials. BMC Musculoskelet Disord 2007; 8:5), and the amplification of this analgesic signal is a novel and unexpected observation.

The analgesic effect observed in patients with OA following IA injection of corticosteroids is likely a result of the suppression of local inflammation (Benito M J, Yeale D J, FitzGerald O, van den Berg W B, Bresnihan B. Synovial tissue inflammation in early and late osteoarthritis. Ann Rheum Dis 2005; 64(9):1263-7; Sellam J, Berenbaum F. The role of synovitis in pathophysiology and clinical symptoms of osteoarthritis. Nat Rev Rheumatol 2010; 6(11):625-35; Wenham C Y, Conaghan P G. The role of synovitis in osteoarthritis. Ther Adv Musculoskelet Dis 2010; 2(6):349-59; Scanzello C R, Goldring S R. The role of synovitis in osteoarthritis pathogenesis. Bone 2012; 51(2): 249-57), and the anti-inflammatory effects of corticosteroids are associated with the repression of inflammatory transcription factors and regulation of post-transcriptional mechanisms. (Newton R. Molecular mechanisms of glucocorticoid action: what is important? Thorax 2000; 55(7):603-13; Barnes P J, Adcock I. Anti-inflammatory actions of steroids: molecular mechanisms. Trends Pharmacol Sci 1993; 14(12):436-41). The long term repression of inflammatory transcription factors and regulation of post-transcriptional mechanisms provides a plausible basis for the analgesic effect of TCA Formulation 1.

For 40 mg TCA Formulation 1, analgesia increased in Weeks 1-4, and plateaued at weeks 5-8; synovial fluid concentration of TCA was 78.75 ng/ml at Week 6. For 10 mg TCA Formulation 1, the same general pattern was observed; analgesia increased in Weeks 1-4, and plateaued after week 5; synovial fluid concentration of TCA was 6.48 ng/ml at Week 6. The maximal analgesic affect provide by the 10 mg dose was less that that provided by the 40 mg dose. Further, the 60 mg dose of TCA Formulation 1 did not provide additional benefit relative to the 40 mg dose (data not shown).

Thus, these studies identify a range of synovial fluid concentration of a corticosteroid that produces maximal analgesic when maintained for a period of approximately 4 weeks post administration. In particular, the achievement and maintenance of maximal analgesic effect requires maintenance of a critical synovial fluid concentration between 6.48 ng/ml and 78.75 ng/ml for a period of approximately 4-6 weeks. Concentrations in excess to the critical concentration will not further amplify the analgesic signal. Further, for periods exceeding approximately 4 weeks, this maximal analgesic effect will persist so long as the critical concentration is maintained. This discovery is a broadly applicable breakthrough with wide-ranging implications. Importantly, the identification of a synovial fluid concentration range for administered corticosteroids is independent of the formulation and/or dosing used, provided that the formulation is effective to deliver the corticosteroid over an extended period of time and to maintain a synovial fluid concentration of corticosteroid in the desired range.

Effects on HPA Axis Function:

The early peak in systemic concentration of TCA produced by TCA IR was not evident at any dose of TCA Formulation 1, and in the first 24 hours following injection, the change from baseline in morning serum cortisol produced by the 10 and 40 mg doses of TCA Formulation 1 was significantly less than that produced by TCA IR. At Week 2, the level of suppression of cortisol by the 10, 40, and 60 mg doses of TCA Formulation 1 and the 40 mg dose of TCA IR were roughly equivalent. At Week 6, the suppression of cortisol produced by each of the treatment arms had returned to near-baseline values.

The 40 and 60 mg doses of TCA Formulation 1 maintained plasma concentrations that were below 1.0 ng/ml but numerically larger than those produced by TCA IR. To understand the impact of prolonged low systemic exposure to TCA produced by TCA Formulation 1 on the HPA axis function, Cumulative Cortisol Suppression (CCS) % values (Meibohm B, Hochhaus G, Mollman H, Barth J, Wagner M, Krieg M, et al. A pharmacokinetic/pharmacodynamic approach to predict the cumulative cortisol suppression of inhaled corticosteroids. Journal of Pharmacokinetics and Biopharmaceutics 1999; 27(2):127-147) were calculated (Table 3) for comparison to published values.

TABLE 3
Median Cumulative Cortisol Suppression following
a single IA injection of 10 mg TCA Formulation 1,
40 mg, 60 mg TCA Formulation 1 and 40 mg TCA IR

| | 10 mg | 40 mg | 60 mg | 40 mg |
|---|---|---|---|---|
| Treatment | Form. 1 | Form. 1 | Form. 1 | TCA IR |
| Day 1-2 | 18.8% | 43.3% | 61.4% | 66.0% |
| Week 2 | 12.5% | 25.6% | 33.7% | 21.4% |
| Week 6 | 10.2% | 16.8% | 4.9% | 6.1% |

On Day 1-2, the 40 mg TCA Formulation 1 and 40 mg TCA IR were associated with CCS % values of 43.3% and 66.0%, respectively; at Week 2, the CCS % values were 25.6% and 21.4%, respectively; and at Week 6 the CCS % values were 16.8% and 6.1%, respectively.

Published CCS % values for corticosteroids chronically administered by oral and inhaled routes provide basis for comparison. BID administration of 20 mg of Methylprednisone produces 80-90% suppression of the HPA axis (Meibohm B, Hochhaus G, Mollman H, Barth J, Wagner M, Krieg M, et al. A pharmacokinetic/pharmacodynamic approach to predict the cumulative cortisol suppression of inhaled corticosteroids. Journal of Pharmacokinetics and Biopharmaceutics 1999; 27(2):127-147); administration at these doses for a period of 3 weeks has the potential to compromise the stress response and induce Cushing's syndrome. In contrast, inhaled steroids (Flunisolide and Fluticasone propionate) are associated with CCS % of 11-40% and have largely benign safety profiles in adults. (Meibohm B, Hochhaus G, Rohatagi S, Mollman H, Barth J, Wagner M, et al. Dependency of cortisol suppression on the administration time of inhaled corticosteroids. J Clin Pharmacol 1997; 37:704-710; Rohatagi S, Bye A, Falcoz C, Mackie A E, Meibohm B, Mollman H et al. Dynamic modeling of cortisol reduction after inhaled administration of fluticasone propionate. J Clin Pharmacol 1996; 36:938-941). At time points at and beyond Week 2, all doses of TCA Formulation 1 produce levels of cortisol suppression that are equal to or less than those produced by Flunisolide and Fluticasone propionate; these data suggest that the persistent low systemic concentrations of TCA associated with TCA Formulation 1 will not have a detrimental effect on cortisol production in patients with intact HPA axis function.

### Example 3. Fluticasone Propionate PLGA Microspheres

Fluticasone propionate PLGA microspheres were produced as described herein. Fluticasone propionate was chosen for these studies due to its very low solubility and that it is approximately 20-fold more potent at the glucocorticosteroid (GC) receptor than other corticosteroids such as TCA. The PLGA used for this study was chosen to provide a slow release profile.

The fluticasone propionate/PLGA microsphere formulations used in in vitro studies included 15% fluticasone propionate (FP) in 75:25 DLG 8E PLGA (75% lactide to 25% glycolide ratio, I.V. of 0.8, ester endcapped PLGA). The fluticasone propionate/PLGA microsphere formulations used in the in vivo studies described below included 40% FP in 75:25 DLG 8E PLGA (75% lactide to 25% glycolide ratio, I.V. of 0.8, ester endcapped PLGA). The inherent viscosity of the FP/PLGA microsphere formulations used in the in vivo studies described below had an inherent viscosity in the range of 0.4 and 0.9 dL/g. Samples were prepared using a spinning disk technique.

This fluticasone propionate/PLGA microsphere formulation used in the in vivo studies described below is referred to herein as the “FP/PLGA formulation.”

The pharmacokinetics of these FP/PLGA formulation were evaluated in a study designed to determine the pharmacokinetic profile in plasma and synovial fluid following a single 5 mg intra-articular injection of the FP/PLGA microsphere formulation. In this study, a total of 20 Beagle dogs were dosed in four groups, with 5 dogs in each group (single sex in each group). Group 1 had synovial fluid collected at 24 hours, Group 2 had synovial fluid collected at 3 months, Group 3 had synovial fluid collected at 4.5 months, and Group 4 had synovial fluid collected at 6 months. Group 4 was terminated at the end of the study, and the joint tissues were processed for histology analysis. Plasma samples from each subject were drawn at the following time points: 1 hr, 4 hr, 24 hr, Day 8, Day 29, Day 92 (3 m), Day 134 (4.5 m), and Day 183 (6 m).

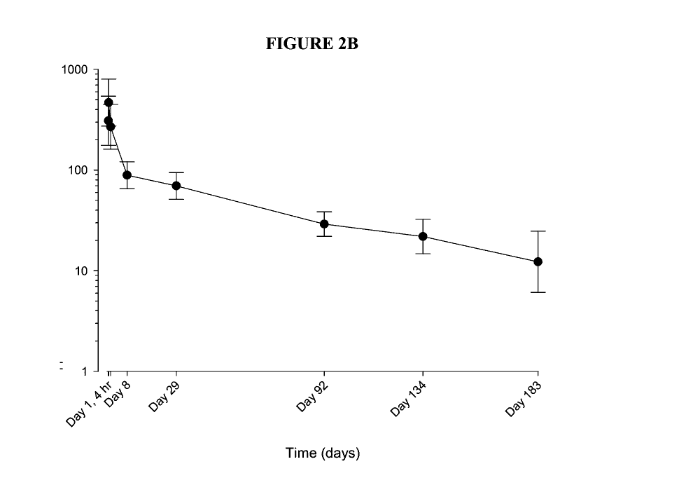

As shown in FIGS. 2A and 2B, sustained plasma levels of fluticasone propionate were detected at least 6 months post-dosing. The FP/PLGA formulation used in this study exhibited favorable PK profile for extended release—this formulation exhibited low Cₘₐₓ, very high AUC_{inf}, and long MRT as shown in Table 4 below:

TABLE 4
PK Profile of FP/PLGA Microspheres
and TCA Formulation 1 Microspheres

| | TCA | TCA | |
|---|---|---|---|
| | Formulation 1 | Formulation 1 | Fluticasone |
| | Single dose | Single dose | Single dose |
| | dog (43 d) - | dog (9 m) - | dog (6 m) - |
| | 6.25 mg dose | 6.25 mg dose | 5 mg dose |
| Cₘₐₓ | 1.04 ± 0.22 | 1.15 ± 0.65 | 0.715 ± 0.464 |
| (ng/mL) | | | |
| AUC₀₋ₗₐₛₜ | 0.8338 ± 0.161 | 5.133 ± 2.422 | 6.632 ± 4.286 |
| (days*ng/mL) | | | |
| AUC_{0-inf} | 8.80 ± 2.54 | 7.57 ± 2.38 | 1736.6 ± 3667.8 |
| (days*ng/mL) | | | |
| MRT₀₋ₗₐₛₜ | 9.8 ± 4.6 | 5.23 ± 2.61 | 30.26 ± 23.90 |
| (days) | | | |
| MRT_{0-inf} | | | 101.31 ± 125.15 |
| (days) | | | |

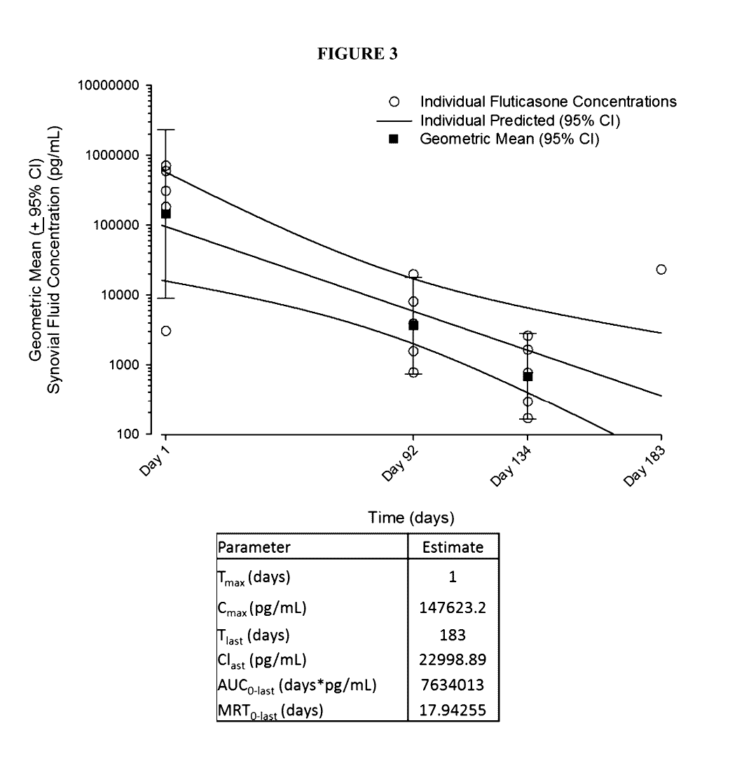

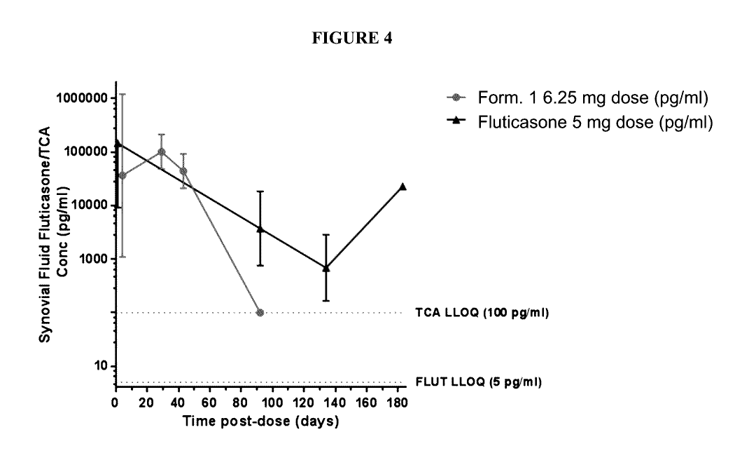

The mean residence time (MRT), a function of the steady-state volume of distribution and time-average clearance obtained from the dose and area under the curve (dose/AUC), was determined for the FP/PLGA formulation. The predicted synovial fluid concentration of fluticasone over time is shown in FIG. 3.

The synovial fluid concentration to plasma concentration ratio for the FP/PLGA formulation is shown in Table 5 below:

TABLE 5
Ratio of Synovial Fluid Concentration of Fluticasone Propionate to Plasma Ratio of
Fluticasone Propionate Over Time

| | | | | Plasma conc. | | Mean |
|---|---|---|---|---|---|---|
| | | Concentration | Mean - | at same time | | Synovial |
| Animal | Study | of SF* | right knee | point as SF | Synovial Fluid:Plasma | Fluid:Plasma |
| ID | Day | (pg/ml) | (pg/ml) | (pg/ml) | Ratio | Ratio |
| 1001A | 1 | 3041.15 | 356717.66 | 40.935 | 74 | 782 |
| 1002A | 1 | 306278.12 | | 470.856 | 650 | |
| 1003A | 1 | 181608.45 | | 231.003 | 786 | |
| 1004A | 1 | 703624.85 | | 484.544 | 1452 | |
| 1005A | 1 | 589035.73 | | 622.919 | 946 | |
| 1106B | 93 | 771.75 | 6764.516 | 43.511 | 18 | 223 |
| 1007B | 93 | 1551.2 | | 30.625 | 51 | |
| 1008B | 92 | 8014.19 | | 29.127 | 275 | |
| 1009B | 92 | 3869.24 | | 28.796 | 134 | |
| 1010B | 92 | 19616.2 | | 30.753 | 638 | |
| 1011C | 134 | 758.94 | 1088.838 | 46.911 | 16 | 42 |
| 1012C | 134 | 1635.32 | | 27.684 | 59 | |
| 1013C | 134 | 167.56 | | 7.08 | 24 | |
| 1014C | 134 | 2591.67 | | 25.344 | 102 | |
| 1015C | 134 | 290.7 | | 42.776 | 7 | |
| 1016D | 183 | 22998.89 | 229988.9 | 19.632 | 1172 | 1172 |

| | | |
|---|---|---|
| BLOQ: below limit of | Mean ratio (all | 400 |
| quantitation (5 pg/mL). | time points) | |

The synovial fluid concentration to plasma concentration ratio for the FP/PLGA formulation is similar to the ratio for TCA Formulation 1 on Day 1, but it decreased over time. The ratio TCA Formulation 1, as compared to a non-extended release TCA formulation (referred to herein as “TCA-IR”) is shown below in Table 6:

TABLE 6
Mean Synovial Fluid Levels of TCA (ng/mL)
from TCA Formulation 1 and TCA IR in Dogs

| Synovial Fluid | TCA IR | TCA Form. 1 | TCA Form. 1 | TCA Form. 1 |
|---|---|---|---|---|
| Level (ng/mL) | 18.75 mg | 2.1 mg | 6.25 mg | 18.75 mg |
| Day 4 | 3677.7 ± 1583.6 | 157.0 ± 97.8 | 666.2 ± 369.0 | 2880.5 ± 1354.5 |
| | (n = 6) | (n = 6) | (n = 7) | (n = 6) |
| Day 29 | 82.2 ± 47.4 | 70.7 ± 18.9 | 138.5 ± 37.7 | 477.8 ± 44.8 |
| | (n = 8) | (n = 8) | (n = 8) | (n = 7) |
| Day 43 | 22.7 ± 19.7 | 27.8 ± 8.2 | 45.6 ± 107.0 | 140.4 ± 39.7 |
| | (n = 4) | (n = 4) | (n = 3) | (n = 3) |

In the previous study summarized in Table 6, most synovial fluid samples from animals sacrificed at 3 months, at 4 months), at 6 months, and at 9 months following single intra-articular doses of TCA Formulation 1 or TCA IR were found to contain either no detectable TCA or levels below the LLOQ (0.1 ng/mL).

The studies presented herein demonstrate that sustained levels of fluticasone were detected at least six months post-dosing with the FP/PLGA formulation.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. Applicants reserve the right to pursue such inventions in later claims.

What is claimed is:

1. A method for maximizing analgesic effect and maintaining maximal analgesic effect in a patient with a disease or disorder associated with joint pain and/or joint inflammation the method comprising:
(a) administering to a subject in need thereof an extended release formulation comprising a corticosteroid; and
(b) maintaining a synovial fluid concentration of the corticosteroid that provides a pharmacological effect equivalent to a synovial fluid concentration of triamcinolone acetonide (TCA) of at least 6 ng/ml for a duration of at least 24 days.

2. The method of claim 1, wherein the synovial fluid concentration of the corticosteroid is maintained at a synovial fluid concentration that provides a pharmacological effect equivalent to a TCA synovial fluid concentration in the range of about 6 ng/ml to about 1000 ng/ml.

3. The method of claim 1, wherein the synovial fluid concentration of the corticosteroid is maintained at a synovial fluid concentration that provides a pharmacological effect equivalent to a TCA synovial fluid concentration in the range of about 6 ng/ml to about 78 ng/ml.

4. The method of claim 1, wherein the synovial fluid concentration of the corticosteroid is maintained for a duration of at least 90 days.

5. The method of claim 1, wherein the synovial fluid concentration of the corticosteroid is maintained for a duration of at least 180 days.

6. The method of claim 1, wherein the synovial fluid concentration of the corticosteroid is maintained for a duration of at least 12 months.

7. The method of claim 1, wherein the synovial fluid concentration of the corticosteroid is maintained by administering at least one additional dose of the corticosteroid.

8. The method of claim 7, wherein the at least one additional dose of the corticosteroid is administered as an extended release formulation.

9. The method of claim 1, wherein the corticosteroid is released from the formulation for a duration of at least between 3 months and 12 months.

10. The method of claim 1, wherein the formulation is administered as one or more injections.

11. The method of claim 10, wherein the injection is one or more local injections at a site of pain.

12. The method of claim 10, wherein the injection is one or more intra-articular or peri-articular injections.

13. The method of claim 1, wherein the disease or disorder associated with joint pain and/or joint inflammation is osteoarthritis, rheumatoid arthritis, acute gouty arthritis, and/or synovitis.

14. The method of claim 1, wherein the corticosteroid is TCA, and the synovial fluid concentration of TCA in the range of about 6 ng/ml to about 1000 ng/ml.

15. The method of claim 1, wherein the corticosteroid is TCA, and the synovial fluid concentration of TCA in the range of about 6 ng/ml to about 78 ng/ml.

16. The method of claim 1, wherein the corticosteroid is ciclesonide (monopropionate), and the synovial fluid concentration of ciclesonide (monopropionate) is in the range of about 168.93 ng/ml to about 28.15 μg/ml.

17. The method of claim 1, wherein the corticosteroid is beclometasone diproprionate, and the synovial fluid concentration of beclometasone diproprionate is in the range of about 32.07 ng/ml to about 5344.96 ng/ml.

18. The method of claim 1, wherein the corticosteroid is dexamethasone, and the synovial fluid concentration of dexamethasone is in the range of about 14.63 ng/ml to about 2438.88 ng/ml.

19. The method of claim 1, wherein the corticosteroid is flunisolide, and the synovial fluid concentration of flunisolide is in the range of about 8.63 ng/ml to about 1438.27 ng/ml.

20. The method of claim 1, wherein the corticosteroid is budesonide, and the synovial fluid concentration of budesonide is in the range of about 1.84 ng/ml to about 307.11 ng/ml.

21. The method of claim 1, wherein the corticosteroid is desisobutyryl-ciclesonide, and the synovial fluid concentration of desisobutyryl-ciclesonide is in the range of about 1.69 ng/ml to about 281.55 ng/ml.

22. The method of claim 1, wherein the corticosteroid is fluticasone propionate, and the synovial fluid concentration of fluticasone propionate is in the range of about 0.95 ng/ml to about 157.58 ng/ml.

23. The method of claim 1, wherein the corticosteroid is mometasone furoate, and the synovial fluid concentration of mometasone furoate is in the range of about 0.77 ng/ml to about 128.93 ng/ml.

24. The method of claim 1, wherein the extended release formulation is a controlled- or sustained-release formulation.

25. A method for maximizing analgesic effect and maintaining maximal analgesic effect in a patient with a disease or disorder associated with joint pain and/or joint inflammation the method comprising:
(a) administering to a subject in need thereof an extended release formulation comprising triamcinolone acetonide (TCA); and
(b) maintaining a synovial fluid concentration of TCA of at least 6 ng/ml for a duration of at least 24 days.

26. The method of claim 25, wherein the synovial fluid concentration of TCA is maintained at a concentration in the range of about 6 ng/ml to about 1000 ng/ml.

27. The method of claim 25, wherein the synovial fluid concentration of TCA is maintained at a concentration in the range of about 6 ng/ml to about 78 ng/ml.

28. The method of claim 25, wherein the synovial fluid concentration of TCA is maintained for a duration of at least 90 days.

29. The method of claim 25, wherein the synovial fluid concentration of TCA is maintained for a duration of at least 180 days.

30. The method of claim 25, wherein the synovial fluid concentration of TCA is maintained for a duration of at least 12 months.

31. The method of claim 25, wherein the synovial fluid concentration of the corticosteroid is maintained by administering at least one additional dose of the corticosteroid.

32. The method of claim 31, wherein the at least one additional dose of the corticosteroid is administered as an extended release formulation.

33. The method of claim 25, wherein the corticosteroid is released from the formulation for a duration of at least between 3 months and 12 months.

34. The method of claim 25, wherein the formulation is administered as one or more injections.

35. The method of claim 34, wherein the injection is one or more local injections at a site of pain.

36. The method of claim 34, wherein the injection is one or more intra-articular or peri-articular injections.

37. The method of claim 25, wherein the disease or disorder associated with joint pain and/or joint inflammation is osteoarthritis, rheumatoid arthritis, acute gouty arthritis, and/or synovitis.

38. The method of claim 25, wherein the extended release formulation is a controlled- or sustained-release formulation.
